(19) United States
(12) Patent Application Publication
(10) Pub. No.: US 2026/0157971 A1
(43) Pub. Date: Jun. 11, 2026

(54) FORMULATIONS AND METHODS FOR TREATING NEUROPSYCHIATRIC DISORDERS

(71) Applicant: HB BIOTECH, INC., New York, NY (US)

(72) Inventors: Francesca Minale, New York, NY (US); Anish Dhanarajan, New York, NY (US); Alon Seifan, New York, NY (US); Tomer Seifan, New York, NY (US)

(73) Assignee: HB BIOTECH, INC., New York, NY (US)

(21) Appl. No.: 19/412,182

(22) Filed: Dec. 8, 2025

Related U.S. Application Data
(63) Continuation of application No. PCT/US2024/032925, filed on Jun. 7, 2024.
(60) Provisional application No. 63/472,213, filed on Jun. 9, 2023.

(51) Int. Cl. A61K 9/24; A61K 9/20; A61K 9/28; A61K 31/277; A61K 31/4184

(52) U.S. Cl. CPC A61K 9/209; A61K 9/2018; A61K 9/2054; A61K 9/2077; A61K 9/2866; A61K 31/277; A61K 31/4184

(57) ABSTRACT

The present disclosure includes formulations and methods for treating brain and/or behavioral health disorders and their associated symptoms. In particular, the present disclosure includes formulations designed to optimize release of the compositions disclosed herein under a wide range of biological conditions after administration.

### CROSS-REFERENCE TO RELATED APPLICATIONS

This application is a continuation under 35 U.S.C. § 111 (a) of PCT International Patent Application No. PCT/US2024/032925, filed Jun. 7, 2024, designating the United States and published in English, which claims priority to and the benefit of U.S. Provisional Application No. 63/472,213, filed Jun. 9, 2023, the entire contents of each of which are incorporated by reference herein.

### BACKGROUND OF THE INVENTION

Brain and behavioral health disorders are the number one cause of disability worldwide. Brain and behavioral health disorders include all of the major disorders that belong to neurology, psychiatry and psychology subspecialties. Anxiety, apathy, cognitive difficulty, depression, fatigue, headache, insomnia, irritability, pain (e.g., body pain), and psychosis are ten cardinal symptoms caused by brain and behavioral health disorders. Individually, these symptoms are disabling features of these disorders, causing loss of function.

All of the ten cardinal symptoms of brain and behavioral health disorders are difficult to treat because most, if not all, people with brain and behavioral disorders suffer from more than one cause of these symptoms. In real clinical practice, the cause of a symptom could be due to neurology, psychiatry, and/or psychological factors. However, patients seek independent treatment for their symptoms. Anxiety, depression, and psychosis are typically treated by a psychiatrist. Headache, pain, and cognitive difficulty is often treated by a neurologist. Irritability, apathy, insomnia and fatigue are often treated by psychologists or primary care doctors. Anxiety is an example of a symptom that is disabling, difficult to treat and multifactorial. Somatic anxiety (hypersensitivity to stimulation) is an example of a neurological cause of anxiety, often related to migraine. Excessive shyness is an example of a psychological cause of anxiety, often related to social phobia. Excessive worry is an example of a psychiatric cause of anxiety, often related to generalized anxiety disorder.

Despite the fact that the ten cardinal disabling symptoms (i.e., anxiety, apathy, cognitive difficulty, depression, fatigue, headache, insomnia, irritability, pain (e.g., body pain), and psychosis) of brain and behavioral health disorders, in real clinical practice, are multi-factorial, current FDA-approved treatments typically target one component of a disorder at a time. For example, selective serotonin reuptake inhibitors (“SSRI's”), which are the first-line treatment for anxiety disorders, were designed specifically to target serotonergic mechanisms of anxiety. The effectiveness of these medications in and of themselves has proven to be limited. Cognitive behavioral therapy is designed specifically to address psychological components of anxiety. Patients receiving both medication and cognitive behavioral therapy are predicted to have better outcomes than those receiving one targeted treatment or the other. Even with cognitive behavioral therapy and medication, treatment success remains limited, suggesting that neurological underpinnings of anxiety remain untreated in the majority of patients seeking relief for anxiety.

No current treatments have been designed to address the multi-factorial features associated with the most disabling symptoms of brain and behavioral health disorders. Accordingly, new treatments that address brain and behavioral health disorders and their symptoms are urgently needed.

### SUMMARY OF THE INVENTION

As described below, the present invention features formulations comprising calcium channel blockers (e.g., anipamil, devapamil, diltiazem, falipamil, felodipine, gallopamil, isradipine, nicardipine, nifedipine, nimodipine, nisoldipine, tiapamil, and verapamil) and angiotensin II receptor blockers (e.g., candesartan or telmisartan) and methods of using such formulations in treating brain and behavioral health disorders.

In one aspect, the present disclosure provides a formulation including a therapeutic combination including a first composition including an effective amount of a first agent that has calcium channel blocking activity, and a second composition including an effective amount of a second agent that has angiotensin II receptor blocking activity. The first composition is formulated for extended release of the first agent when administered to a subject and the second composition is formulated for immediate release of the second agent when administered to the subject.

In another aspect, the present disclosure provides a formulation including a therapeutic combination including a first composition including an effective amount of a first agent that has calcium channel blocking activity, and a second composition including an effective amount of a second agent that has angiotensin II receptor blocking activity. The second composition is formulated to have a faster rate of release of the second agent compared to the rate of release of the first agent for the first composition when administered to a subject.

In another aspect, the present disclosure provides a formulation including a therapeutic combination including a first composition including an effective amount of a first agent that has calcium channel blocking activity, and a second composition including an effective amount of a second agent that has angiotensin II receptor blocking activity. The second composition is formulated for immediate release and dissolution of the second agent when in contact with a solution having a pH between 1-5.

In another aspect, the present disclosure provides a formulation including a therapeutic combination including a first composition including an effective amount of a first agent that has calcium channel blocking activity, and a second composition including an effective amount of a second agent that has angiotensin II receptor blocking activity. The second composition is formulated for immediate release and dissolution of the second agent under fed gastric conditions or intestinal conditions in a subject when administered to the subject.

In another aspect, the present disclosure provides a tablet including a therapeutic combination including a first composition including an effective amount of a first agent that has calcium channel blocking activity, and a second composition including an effective amount of a second agent that has angiotensin II receptor blocking activity. The second composition is formulated to release the second agent before the first composition releases the first agent when the tablet is administered to a subject.

In another aspect, the present disclosure provides a compressed tablet including the formulations of any of the above aspects, or embodiments thereof.

In another aspect, the present disclosure provides a press coated tablet including the formulations of any of the above aspects, or embodiments thereof.

In another aspect, the present disclosure provides a capsule including the formulations of any of the above aspects, or embodiments thereof.

In another aspect, the present disclosure provides a pharmaceutical composition including the formulation of any of the above aspects, or embodiments thereof, and a pharmaceutically acceptable excipient.

In another aspect, the present disclosure provides a push-pull osmotic pump tablet including the formulation any of the above aspects, or embodiments thereof. The osmotic pump tablet further includes a first layer including the first composition, a second layer including the second composition, and a semipermeable coating. The first layer further includes a polymeric osmogene. The osmotic pump tablet is configured such that the second agent is released through burst release and the first agent is released through leaching through the semipermeable coating when the osmotic pump tablet is contacted with an aqueous solution.

In another aspect, the present disclosure provides a method for treating a subject having a brain or behavioral health disorder. The method involves administering to the subject the formulations of any of the above aspects, or embodiments thereof, the tablets of any of the above aspects, or embodiments thereof, the compressed tablet of any of the above aspects, or embodiments thereof, the press coated tablet of any of the above aspects, or embodiments thereof, the capsules of any of the above aspects, or embodiments thereof, the pharmaceutical compositions of any of the above aspects, or embodiments thereof, or the push-pull osmotic pump tablet of any of the above aspects, or embodiments thereof, thereby treating the brain or behavioral health disorder.

In another aspect, the present disclosure provides a method for treating an anxiety disorder. The method involves administering to the subject the formulations of any of the above aspects, or embodiments thereof, the tablets of any of the above aspects, or embodiments thereof, the compressed tablet of any of the above aspects, or embodiments thereof, the press coated tablet of any of the above aspects, or embodiments thereof, the capsules of any of the above aspects, or embodiments thereof, the pharmaceutical compositions of any of the above aspects, or embodiments thereof, or the push-pull osmotic pump tablet of any of the above aspects, or embodiments thereof, thereby treating the anxiety disorder.

In another aspect, the present disclosure provides a method of increasing advancement of a subject along Maslow's hierarchy of needs. The method involves administering to the subject the formulations of any of the above aspects, or embodiments thereof, the tablets of any of the above aspects, or embodiments thereof, the compressed tablet of any of the above aspects, or embodiments thereof, the press coated tablet of any of the above aspects, or embodiments thereof, the capsules of any of the above aspects, or embodiments thereof, the pharmaceutical compositions of any of the above aspects, or embodiments thereof, or the push-pull osmotic pump tablet of any of the above aspects, or embodiments thereof, thereby increasing advancement of the subject along Maslow's hierarchy of needs. The first agent alters metabolism and/or blood flow associated with the adrenergic system and the second agent alters metabolism and/or blood flow associated with the brain renin angiotensin aldosterone system. The increase in advancement is relative to a reference.

In another aspect, the present disclosure provides a kit including the formulations of any of the above aspects, or embodiments thereof, the tablets of any of the above aspects, or embodiments thereof, the compressed tablet of any of the above aspects, or embodiments thereof, the press coated tablet of any of the above aspects, or embodiments thereof, the capsules of any of the above aspects, or embodiments thereof, the pharmaceutical compositions of any of the above aspects, or embodiments thereof, or the push-pull osmotic pump tablet of any of the above aspects, or embodiments thereof, and instructions for the use of the formulation, tablet, compressed tablet, press coated tablet, capsule, pharmaceutical composition, or push-pull osmotic pump tablet for the treatment of a brain and/or behavioral health disorder or a symptom thereof.

In any of the above aspects, or embodiments thereof, the first agent is anipamil, devapamil, diltiazem, falipamil, felodipine, gallopamil, isradipine, nicardipine, nifedipine, nimodipine, nisoldipine, tiapamil, or verapamil.

In any of the above aspects, or embodiments thereof, the second agent is telmisartan or candesartan.

In any of the above aspects, or embodiments thereof, the combination is labeled for the treatment of a brain and/or behavioral health disorder.

In any of the above aspects, or embodiments thereof, the combination is labeled for the treatment of a symptom of a brain and/or behavioral health disorder. The symptom of a brain and/or behavioral health disorder is anxiety, apathy, cognitive difficulty, depression, fatigue, headache, insomnia, irritability, pain or psychosis.

In any of the above aspects, or embodiments thereof, the first agent is verapamil.

In any of the above aspects, or embodiments thereof, the second agent is telmisartan.

In any of the above aspects, or embodiments thereof, the second composition is formulated such that greater than 80% of the second agent is released within 1 hour of administration to a subject.

In any of the above aspects, or embodiments thereof, the second composition is formulated such that greater than 95% of the second agent is released within 2 hours of administration to a subject.

In any of the above aspects, or embodiments thereof, the second composition is formulated such that greater than 85% of the second agent is released within 1.5 hours of administration to a subject.

In any of the above aspects, or embodiments thereof, the first composition is formulated for release of the first agent over a period of 24 hours.

In any of the above aspects, or embodiments thereof, the effective amount of the first agent is from about 10 mg to about 720 mg and the effective amount of the second agent is from about 2 mg to about 240 mg.

In any of the above aspects, or embodiments thereof, the effective amount of the first agent is from about 60 mg to about 360 mg and the effective amount of the second agent is from about 20 mg to about 180 mg.

In any of the above aspects, or embodiments thereof, the effective amount of the first agent is from about 10 mg to about 120 mg and the effective amount of the second agent is from about 1 mg to about 40 mg.

In any of the above aspects, or embodiments thereof, the effective amount of the first agent is about 15 mg and the effective amount of the second agent is about 2.5 mg, or the effective amount of the first agent is about 30 mg and the effective amount of the second agent is about 5 mg.

In any of the above aspects, or embodiments thereof, the effective amount of the first agent is about 288 mg and the effective amount of the second agent is about 96 mg.

In any of the above aspects, or embodiments thereof, the mass ratio of the first agent to the second agent is from about 2:1 to about 5:1.

In any of the above aspects, or embodiments thereof, the mass ratio of the first agent to the second agent is about 2:1.

In any of the above aspects, or embodiments thereof, the amount of first agent is from about 1 to about 4 times the amount of the second agent.

In any of the above aspects, or embodiments thereof, the amount of first agent is about 2 times the amount of the second agent.

In any of the above aspects, or embodiments thereof, the formulation or tablet further includes magnesium oxide.

In any of the above aspects, or embodiments thereof, the formulation or tablet further includes at least about 150 mg magnesium oxide.

In any of the above aspects, or embodiments thereof, the second composition further includes sodium hydroxide.

In any of the above aspects, or embodiments thereof, the sodium hydroxide is present in an amount necessary to dissolve the second agent when administered to a subject.

In any of the above aspects, or embodiments thereof, the first composition further includes a hydrophilic matrix.

In any of the above aspects, or embodiments thereof, the hydrophilic matrix includes hydroxypropyl methylcellulose.

In any of the above aspects, or embodiments thereof, the second composition is formulated to form a coating around the first composition.

In any of the above aspects, or embodiments thereof, each of the first composition and the second composition are formulated as distinct layers.

In any of the above aspects, or embodiments thereof, the first composition is formulated in tablet form and the second composition is formulated in particle form. The first composition and the second composition are pressed together to form the compressed tablet.

In any of the above aspects, or embodiments thereof, each of the first composition and the second composition are formulated as pellets, particles, or granules.

In any of the above aspects, or embodiments thereof, the press coated tablet includes the first composition surrounded by a compressed layer of the second composition. The first composition is formulated as a tablet, and the second composition is formulated as a powder.

In any of the above aspects, or embodiments thereof, the first composition is formulated as a tablet and the second composition is formulated as pellets, particles, granules, or a powder.

In any of the above aspects, or embodiments thereof, each of the first composition and the second composition is formulated as a tablet.

In any of the above aspects, or embodiments thereof, the pharmaceutical composition further includes magnesium oxide.

In any of the above aspects, or embodiments thereof, the formulation, tablet, compressed tablet, press coated tablet, capsule, pharmaceutical composition, or push-pull osmotic pump tablet is formulated to increase bioavailability of the second agent under fed conditions when administered to a subject.

In any of the above aspects, or embodiments thereof, the formulation, tablet, compressed tablet, press coated tablet, capsule, pharmaceutical composition, or push-pull osmotic pump tablet is administered once daily.

In any of the above aspects, or embodiments thereof, the formulation, tablet, compressed tablet, press coated tablet, capsule, pharmaceutical composition, or push-pull osmotic pump tablet is administered twice daily.

In any of the above aspects, or embodiments thereof, the administration is associated with an alteration in cerebral metabolism or cerebral blood flow.

In any of the above aspects, or embodiments thereof, cerebral blood flow is altered in one or more of the telencephalon, the diencephalon, and the mesencephalon.

In any of the above aspects, or embodiments thereof, the alteration in regional cerebral blood flow is associated with the establishment of hemodynamic equilibrium in a region of the brain.

In any of the above aspects, or embodiments thereof, the method increases a primary outcome, where the primary outcome is cognitive function, life satisfaction, the subject's sense of meaning and purpose, the subject's sense of emotional or instrumental support, friendship, or life satisfaction.

In any of the above aspects, or embodiments thereof, the method further includes administering magnesium oxide to the subject.

In any of the above aspects, or embodiments thereof, at least about 150 mg magnesium oxide is administered to the subject daily.

The invention provides formulations comprising calcium channel blockers (e.g., verapamil) and angiotensin II receptor blockers (e.g., candesartan or telmisartan) and methods of using such formulations in treating brain and behavioral health disorders. Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound. Exemplary agents include but are not limited to anipamil, candesartan, devapamil, diltiazem, falipamil, felodipine, gallopamil, isradipine, nicardipine, nifedipine, nimodipine, nisoldipine, telmisartan, tiapamil, and verapamil.

By “alteration” is meant a positive or negative change. As used herein, an alteration includes a 5%, 10%, 15%, 20%, 25%, 30%, 35%, 45%, 50%, 55%, 60%, 65%, 70%, 75%, 80%, 85%, 90%, 95%, or 100% change. The change can be an increase or a reduction in an analyte or clinical indicator. For example, the measured quantity can in regional cerebral blood flow. The measured clinical indicator can be a quantitative assessment of the magnitude of a symptom of a disease (e.g., anxiety, apathy, cognitive difficulty, depression, fatigue, headache, insomnia, irritability, pain (e.g., body pain), or psychosis). In some embodiments, an alteration is a reduction in a symptom associated with a brain and/or behavioral health disorder. In some embodiments, an alteration is an increase in function associated with the treatment of a brain and/or behavioral health disorder.

By “ameliorate” is meant decrease, suppress, attenuate, diminish, arrest, or stabilize the development or progression of a disease or disorder. In various embodiments, the disease or disorder is a brain disease or behavioral health disorder (e.g., affective disorders, anxiety disorders, neurodegenerative disorders, neurodevelopmental disorders, psychotic disorders, personality disorders, migraine disorders, somatoform disorders, substance use disorders, seizure disorders, neuroimmune disorders and cerebrovascular disorders. Examples of affective disorders include bipolar disorder, cyclothymia, depression, dysthymia, cyclothymia, major depressive disorder, postpartum depression, and seasonal affective disorder. Examples of anxiety disorders include generalized anxiety disorder, panic disorder, social anxiety disorder, post-traumatic stress disorder, obsessive compulsive disorder, and specific phobia).

By “analog” is meant a molecule that is not identical to a molecule of interest, but that has analogous functional and/or structural features. In some embodiments, an analog is an agent that targets the adrenergic system. In some embodiments, an analog is an agent that targets the renin angiotensin aldosterone system.

By “anipamil” is meant a compound having the following structure:

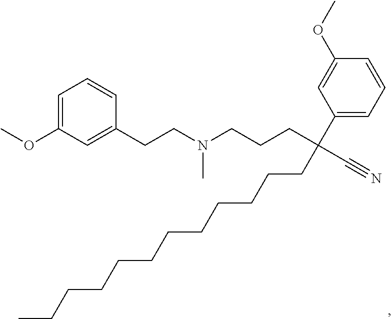 [SMILES: CCCCCCCCCCCCC(C#N)(CCCN(C)CCc1cccc(OC)c1)c1cccc(OC)c1]

or a pharmaceutically acceptable salt thereof. In some embodiments, anipamil has calcium channel blocking activity.

By “candesartan” is meant a compound having the following structure:

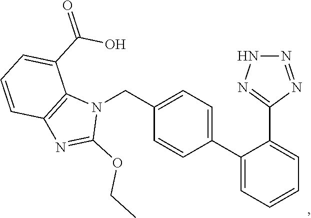 [SMILES: CCOc1nc2cccc(C(=O)O)c2n1Cc1ccc(-c2ccccc2-c2nn[nH]n2)cc1]

or a pharmaceutically acceptable salt thereof. In some embodiments, candesartan has angiotensin II receptor blocking activity.

By “cerebral metabolism” is meant the rate of metabolism in the brain or a region thereof. Cerebral metabolism is measured using any of a variety of methods available to a practitioner including, as non-limiting examples thereof, X-ray computed tomography (CT), positron emission tomography (PET), near-infrared spectroscopy (NIRS), magnetic resonance imaging (MRI), and those methods provided herein. In embodiments, an increase in metabolism in a region of the brain is associated with an increase in blood flow to the region. Cerebral blood flow is measured using any of a variety of methods available to a practitioner including, as non-limiting examples thereof, single-photon emission computed tomography (SPECT), positron emission tomography (PET), functional MRI (fMRI), arterial spin labeling (ASL) MRI, transcranial Doppler ultrasound imaging (i.e., sonography), phase-contrast MRI, and near-infrared spectroscopy (NIRS).

In this disclosure, “comprises,” “comprising,” “containing,” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

By “decrease” is meant a negative alteration.

By “devapamil” is meant a compound having the following structure:

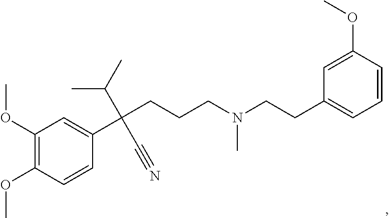 [SMILES: COc1cccc(CCN(C)CCCC(C#N)(c2ccc(OC)c(OC)c2)C(C)C)c1]

or a pharmaceutically acceptable salt thereof. In an embodiment, devapamil has calcium channel blocking activity.

By “diltiazem” is meant a compound having the following structure:

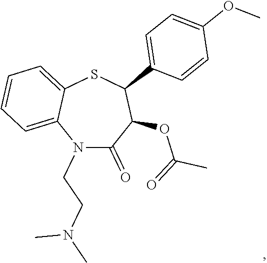 [SMILES: COc1ccc([C@@H]2Sc3ccccc3N(CCN(C)C)C(=O)[C@@H]2OC(C)=O)cc1]

or a pharmaceutically acceptable salt thereof. In some embodiments, diltiazem has calcium channel blocking activity.

By “disease” is meant any condition or disorder that damages or interferes with the normal function of a cell, tissue, or organ. In embodiments, the disease or disorder is a neuropsychiatric disorder, examples of which include brain and/or behavioral health disorders and symptoms thereof. Non-limiting examples of brain and behavioral health disorders include affective disorders, anxiety disorders, neurodegenerative disorders, neurodevelopmental disorders, psychotic disorders, personality disorders, migraine disorders, somatoform disorders, substance use disorders, seizure disorders, neuroimmune disorders and cerebrovascular disorders. Examples of affective disorders include bipolar disorder, cyclothymia, dysthymia, major depressive disorder, postpartum depression, and seasonal affective disorder. Examples of anxiety disorders include generalized anxiety disorder, panic disorder, social anxiety disorder, post-traumatic stress disorder, obsessive compulsive disorder, and specific phobia. Examples of neurodegenerative disorders include Alzheimer's disease, Parkinson's disease and Dementia with Lewy Bodies. Examples of neurodevelopmental disorders include autism spectrum disorder, attention deficit hyperactive disorder (ADHD) and learning disorders. Examples of psychotic disorders include schizophrenia, schizoaffective disorder, and major depression with psychosis. Examples of personality disorders include paranoid, schizoid, schizotypal, antisocial, borderline, histrionic, narcissistic, avoidant, dependent, and obsessive-compulsive personality disorder. Examples of migraine-related disorders include migraine with aura, migraine without aura, acephalgic migraine, and vestibular migraine. Examples of somatoform disorders include somatization disorder, hypochondriasis, conversion disorder, body dysmorphic disorder and chronic pain. Examples of substance use disorders include alcohol use disorder and opioid use disorder. Examples of seizure disorders include epileptic disorders and psychogenic non-epileptic seizure disorders. Examples of neuroimmune disorders include multiple sclerosis. Examples of cerebrovascular disorders include cerebrovascular disease, vascular dementia and cerebrovascular accident. The symptoms associated with the disease are selected from one or more of the “ten cardinal symptoms” associated with brain and behavioral health disorders, including but not limited to anxiety, apathy, cognitive difficulty, depression, fatigue, headache, insomnia, irritability, pain (e.g., body pain), and psychosis. In embodiments, the disease is associated with altered regional cerebral blood flow relative to a reference (e.g., the blood flow present in a healthy control brain). The disease can be a brain or behavioral health disorder as defined above.

By “effective amount” is meant an amount of an agent sufficient to treat a disease or disorder. In one embodiment, an effective amount of a combination therapy described herein is sufficient to treat a brain and/or behavioral disorder or a symptom thereof, or to effect an improvement in a primary outcome (e.g., increase cognitive function, life satisfaction, the subject's sense of meaning and purpose, the subject's sense of emotional or instrumental support, friendship, and life satisfaction).

By “falipamil” is meant a compound having the following structure:

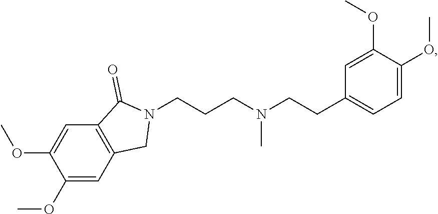

or a pharmaceutically acceptable salt thereof. In some embodiments, falipamil has calcium channel blocking activity.

By “felodipine” is meant a compound having the following structure:

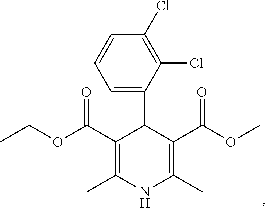 [SMILES: CCOC(=O)C1=C(C)NC(C)=C(C(=O)OC)C1c1cccc(Cl)c1Cl]

or a pharmaceutically acceptable salt thereof. In some embodiments, felodipine has calcium channel blocking activity.

By “gallopamil” is meant a compound having the following structure:

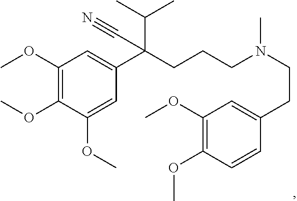 [SMILES: COc1ccc(CCN(C)CCCC(C#N)(c2cc(OC)c(OC)c(OC)c2)C(C)C)cc1OC]

or a pharmaceutically acceptable salt thereof. In some embodiments, gallopamil has calcium channel blocking activity.

By “hemodynamic equilibrium” is meant a state of balance with respect to relative blood flow rates among corresponding regions of the brain. In embodiments, hemodynamic equilibrium is associated with approximately equal relative blood flow rates and/or metabolic activity among the regions.

By “increases” is meant a positive alteration.

By “isradipine” is meant a compound having the following structure:

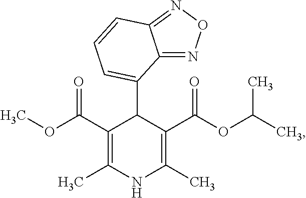 [SMILES: COC(=O)C1=C(C)NC(C)=C(C(=O)OC(C)C)C1c1cccc2nonc12]

or a pharmaceutically acceptable salt thereof. In some embodiments, isradipine has calcium channel blocking activity.

By “nicardipine” is meant a compound having the following structure:

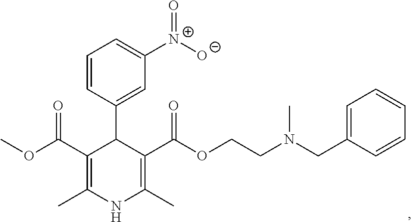 [SMILES: COC(=O)C1=C(C)NC(C)=C(C(=O)OCCN(C)Cc2ccccc2)C1c1cccc([N+](=O)[O-])c1]

or a pharmaceutically acceptable salt thereof. In some embodiments, nicardipine has calcium channel blocking activity.

By “nifedipine” is meant a compound having the following structure:

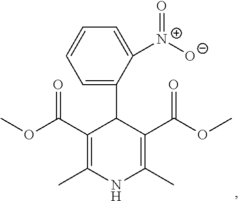

or a pharmaceutically acceptable salt thereof. In some embodiments, nifedipine has calcium channel blocking activity.

By “nimodipine” is meant a compound having the following structure:

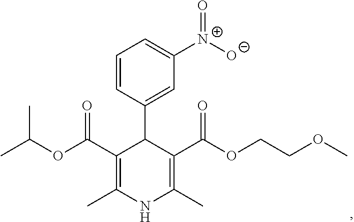 [SMILES: COCCOC(=O)C1=C(C)NC(C)=C(C(=O)OC(C)C)C1c1cccc([N+](=O)[O-])c1]

or a pharmaceutically acceptable salt thereof. In some embodiments, nimodipine has calcium channel blocking activity.

By “nisoldipine” is meant a compound having the following structure:

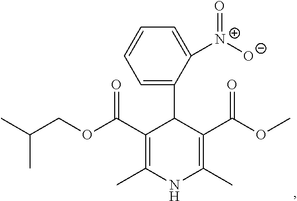 [SMILES: COC(=O)C1=C(C)NC(C)=C(C(=O)OCC(C)C)C1c1ccccc1[N+](=O)[O-]]

or a pharmaceutically acceptable salt thereof. In some embodiments, nisoldipine has calcium channel blocking activity.

As used herein, “obtaining” as in “obtaining an agent” includes synthesizing, purchasing, or otherwise acquiring the agent.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reduces” is meant a negative alteration.

By “subject” is meant a mammal. Non-limiting examples of mammals include a human or non-human mammal, such as a bovine, equine, canine, ovine, feline, or rodent.

By “reference” is meant a standard or control condition. In one embodiment, the effect of an agent on a cell is compared to the effect of the agent on a control cell. In embodiments, the reference is a healthy subject. In embodiments, a clinical feature of subject having a brain or behavioral disorder is compared to a reference clinical feature present in a healthy subject. The healthy subject is a subject not having a disorder or condition of interest. In some embodiments, the reference is an untreated patient or a subject prior to treatment or prior to an alteration in treatment.

By “region of the brain” is meant a portion of the brain. In embodiments, the portion of the brain contains one or more of the telencephalon, the diencephalon, and the mesencephalon. The region can contain all three of the telencephalon, the diencephalon, and the mesencephalon.

By “simultaneous administration” is meant administering concurrently.

By “sequential administration” is meant administered at separate points in time. For example, one or more agents are administered sequentially if the administration is separated by minutes, hours, or days. For example, in sequential administration a first agent is administered 15, 30, 45, or 60 minutes prior to the administration of one or more additional agents. In another example of sequential administration, a first agent is administered 1, 2, 3, 4, 5, 6, 12, or 24 hours prior to the administration of the second agent. In yet another example of sequential administration, a first agent is administered 1, 2, 3, 4, 5, 6, or 7 days prior to the administration of the second agent.

By “telmisartan” is meant a compound having the following structure:

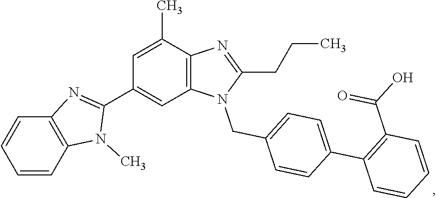 [SMILES: CCCc1nc2c(C)cc(-c3nc4ccccc4n3C)cc2n1Cc1ccc(-c2ccccc2C(=O)O)cc1]

or a pharmaceutically acceptable salt thereof. In some embodiments, telmisartan has angiotensin II receptor blocking activity.

“Therapeutic agent” means a substance that has the potential of affecting the function of an organism. Such a compound is, for example, a naturally occurring, semi-synthetic, or synthetic agent. For example, an agent is a drug that targets a specific function of an organism. A therapeutic agent can decrease, suppress, attenuate, diminish, arrest, or stabilize the development or progression of disease, disorder, or condition. A therapeutic agent is associated with an alteration in regional cerebral blood flow in a subject. Non-limiting examples of therapeutic agents described herein include anipamil, candesartan, devapamil, diltiazem, falipamil, felodipine, gallopamil, isradipine, nicardipine, nifedipine, nimodipine, nisoldipine, telmisartan, tiapamil, and verapamil, as well as derivatives, analogs, and functional equivalents of such agents. In embodiments, a therapeutic combination features a first agent selected from one or more of anipamil, devapamil, diltiazem, falipamil, felodipine, gallopamil, isradipine, nicardipine, nifedipine, nimodipine, nisoldipine, tiapamil, and verapamil and a second agent selected from one or more of candesartan and telmisartan.

By “effective amount” is meant the amount of an agent required to reduce or ameliorate a symptom of a disease relative to a reference. The effective amount of active compound(s) (e.g., anipamil, candesartan, devapamil, diltiazem, falipamil, felodipine, gallopamil, isradipine, nicardipine, nifedipine, nimodipine, nisoldipine, telmisartan, tiapamil, and verapamil) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. The symptom is selected from one or more of “ten cardinal symptoms” associated with brain and behavioral health disorders: anxiety, apathy, cognitive difficulty, depression, fatigue, headache, insomnia, irritability, pain (e.g., body pain), and psychosis. In embodiments, the therapeutically effective amount is the amount of an agent or combination of agents necessary

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “tiapamil” is meant a compound having the following structure:

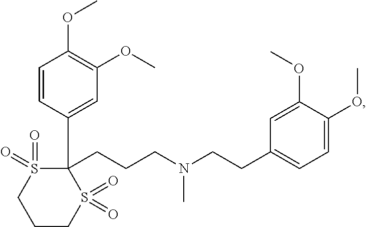 [SMILES: COc1ccc(CCN(C)CCCC2(c3ccc(OC)c(OC)c3)S(=O)(=O)CCCS2(=O)=O)cc1OC]

or a pharmaceutically acceptable salt thereof. In some embodiments, tiapamil has calcium channel blocking activity.

As used herein, the terms “treat,” treating,” “treatment,” and the like refer to reducing or ameliorating a disease and/or symptoms associated therewith. It will be appreciated that, although not precluded, treating a disorder or condition does not require that the disorder, condition or symptoms associated therewith be completely eliminated.

By “verapamil” is meant an agent having the following structure:

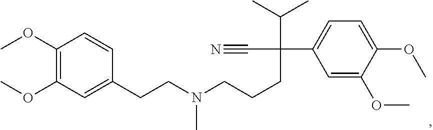

or a pharmaceutically acceptable salt thereof. In some embodiments, verapamil has calcium channel blocking activity.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein. Where various mechanisms of action, hypotheses, or theories are discussed throughout the application, these are not intended to be limiting.

### BRIEF DESCRIPTION OF THE DRAWINGS

FIG. 1 provides an illustration of a 24-hour release formulation of telmisartan and verapamil.

FIG. 2 is a graph showing dissolution test results for verapamil in: i) 0.1N HCl; ii) pH 4.5; and iii) pH 6.8, in a 24-hour release formulation of telmisartan and verapamil.

FIG. 3 is a graph showing dissolution test results for telmisartan in: i) 0.1N HCl; ii) pH 4.5; and iii) pH 6.8, in a 24-hour release formulation of telmisartan and verapamil.

FIG. 4 provides an illustration of an example biphasic formulation of telmisartan and verapamil.

FIG. 5 is a graph showing dissolution test results for verapamil in: i) 0.1N HCl; ii) pH 4.5; and iii) pH 6.8, in the biphasic formulation of FIG. 4.

FIG. 6 is a graph showing dissolution test results for telmisartan in 0.1N HCl, in the biphasic formulation of FIG. 4.

FIG. 7 provides an illustration of an example alternate formulation of telmisartan and verapamil.

FIG. 8 provides an illustration of an example alternate formulation of telmisartan and verapamil.

FIG. 9 provides an illustration of an example alternate formulation of telmisartan and verapamil.

FIG. 10 provides an illustration of an example alternate formulation of telmisartan and verapamil.

FIG. 11 provides an illustration of an example alternate formulation of telmisartan and verapamil.

FIG. 12 provides an illustration of an example alternate formulation of telmisartan and verapamil.

### DETAILED DESCRIPTION OF THE INVENTION

The invention features formulations that are useful for the treatment of brain and/or behavioral health disorders, and their associated symptoms.

The invention is based, at least in part, on the discovery of a combination therapy formulation of telmisartan and verapamil that provides for the immediate release of telmisartan under conditions that simulated gastric pH, while verapamil was released over a 24-hour timeframe in all pH conditions. This formulation was achieved by adding an immediate release coating of telmisartan on the outside of the verapamil-controlled release matrix tablet.

Accordingly, the disclosure provides formulations containing telmisartan or candesartan that can be combined with other agents that target the adrenergic system (e.g., anipamil, devapamil, diltiazem, falipamil, felodipine, gallopamil, isradipine, nicardipine, nifedipine, nimodipine, nisoldipine, tiapamil, and verapamil) for the treatment of brain and/or behavioral health disorders and their associated symptoms, using the formulations disclosed herein. In some embodiments, the formulations of the combination therapy disclosed herein may increase bioavailability of an agent that targets the adrenalin and brain renin angiotensin aldosterone systems (RAAS), such as telmisartan, as compared to a standard formulation of the combination therapy, such as a 24-hour release formulation.

### Brain and Behavioral Disorders

Collectively, brain and behavioral disorders are the number one cause of disability worldwide. Achieving comprehensive remission, across the spectrum of so many different symptoms, is difficult for most patients, doctors and families because in real clinical practice, patients have multiple co-morbid diagnoses. Treating a patient with multiple co-morbidities requires separate individual treatment for each disorder, which increases complexity, inflates cost and lowers safety of care.

Brain and behavioral health disorders, at least partially due to having multiple components, result in serious psychosocial issues that negatively impact a patient's quality of life. However, current treatments are designed to treat only a single (e.g., psychiatric) component.

Although brain and behavioral health disorders are a clinically diverse group of disorders, abnormal functional connectivity represents a shared, common pathological framework. Regardless of the disorder, two independent systems, the adrenergic system and the brain renin angiotensin aldosterone system, directly influence functional connectivity. Among these different disorders, abnormalities within similar functional networks contribute to symptomatology. For example, anxiety disorders and chronic pain can be classified, together, as reactive disorders, each featuring atypical connectivity between sensorimotor and salience networks. In fact, successful treatment of symptoms for many brain and behavioral disorders has been shown to be associated with normalization of atypical patterns of regional cerebral blood flow.

Non-limiting examples of brain and behavioral health disorders include affective disorders, anxiety disorders, neurodegenerative disorders, neurodevelopmental disorders, psychotic disorders, personality disorders, migraine disorders, somatosensory somatoform disorders, substance use disorders, seizure disorders, neuroimmune disorders and cerebrovascular disorders. Examples of affective disorders include bipolar disorder, cyclothymia, dysthymia, major depressive disorder, postpartum depression, and seasonal affective disorder. Examples of anxiety disorders include generalized anxiety disorder, panic disorder, social anxiety disorder, post-traumatic stress disorder, obsessive compulsive disorder and specific phobia. Examples of neurodegenerative disorders include Alzheimer's disease, Parkinson's disease and Dementia with Lewy Bodies. Examples of neurodevelopmental disorders include autism spectrum disorder, attention deficit hyperactive disorder (ADHD) and learning disorders. Examples of psychotic disorders include schizophrenia, schizoaffective disorder and major depression with psychosis. Examples of personality disorders include paranoid, schizoid, schizotypal, antisocial, borderline, histrionic, narcissistic, avoidant, dependent and obsessive-compulsive personality disorder. Examples of migraine disorders include migraine with aura, migraine without aura, acephalgic migraine, and vestibular migraine. Examples of somatoform disorders include somatization disorder, hypochondriasis, conversion disorder, body dysmorphic disorder and chronic pain. Examples of substance use disorders include alcohol use disorder and opioid use disorder. Examples of seizure disorders include epileptic disorders and psychogenic non-epileptic seizure disorders. Examples of neuroimmune disorders include multiple sclerosis. Examples of cerebrovascular disorders include cerebrovascular disease, vascular dementia and cerebrovascular accident. The symptoms associated with the disease is selected from one or more of the “ten cardinal symptoms” associated with brain and behavioral health disorders, including but not limited to anxiety, apathy, cognitive difficulty, depression, fatigue, headache, insomnia, irritability, body pain and psychosis. Such symptoms are measured using any of a variety of methods available to a practitioner, non-limiting examples of which are presented in Brenes, “Anxiety, Depression, and Quality of Life in Primary Care Patients”, Prim Care Companion J Clin Psychiatry, 9:437-443 (2007) and in Julian, “Measures of Anxiety”, Arthritis Care Res, 63:1-11 (2011).

No current treatments for the above listed disorders are designed to address the functional connectivity abnormalities that are germane to the disorders. Selective serotonin reuptake inhibitors (“SSRI's”) have limited effectiveness for anxiety. Even with cognitive behavioral therapy and medication, treatment success remains limited. Since SSRI's do not treat symptoms caused by common anxiety co-morbidities such as ADHD, disability due to co-morbidities remains untreated in the majority of patients seeking relief for anxiety. For patients with ADHD, stimulants are not effective for, and can often exacerbate, co-morbid mood disorders. These are critical treatment gaps because the symptoms caused by psychiatric co-morbidities are often the most disabling. Moreover, current controlled treatment options, specifically benzodiazepines, stimulants and opiates, are dangerous due to exacerbation of co-morbid mood disorders, addiction and lethal overdose.

### Combination Therapies

Certain existing pharmaceuticals that influence the adrenergic system and the brain renin angiotensin aldosterone system and never previously used in combination can be combined to create novel, synergistic changes in cerebral blood flow that translate into desirable clinical outcomes. Combination treatments of the present invention involve, for example, administering to a subject a combination of two or more agents selected from previously FDA-approved cardiovascular medications. The invention features combinations containing verapamil and telmisartan, verapamil and candesartan, as well as candesartan or telmisartan in combination with one or more of anipamil, devapamil, diltiazem, falipamil, felodipine, gallopamil, isradipine, nicardipine, nifedipine, nimodipine, nisoldipine, and tiapamil verapamil.

Individually, agents are selected to target the renin angiotensin aldosterone system and the adrenergic system. Telmisartan and candesartan, individually or in combination, balance the severity of the angiotensin response because they block angiotensin 2 receptors. Not wishing to be bound by theory, anipamil, devapamil, diltiazem, falipamil, felodipine, gallopamil, isradipine, nicardipine, nifedipine, nimodipine, nisoldipine, tiapamil, and verapamil, individually or in combination, balance the severity of the adrenalin response because they influence output of norepinephrine from the midbrain. Since both the adrenergic system and the brain RAAS interact to modulate functional connectivity, combining agents that target each system independently creates synergistic effects resulting in novel treatment effects.

A novel treatment is presented herein that ameliorates the psychiatric, psychological and neurological components of disorders that manifest as one or more of the ten cardinal neuro-psychiatric symptoms (i.e., anxiety, apathy, cognitive difficulty, depression, fatigue, headache, insomnia, irritability, pain (e.g., body pain), and psychosis), regardless of the underlying diagnosis. Without intending to be bound by theory, compositions and/or dosage forms of the present invention containing a therapeutic combination (e.g., verapamil and telmisartan, verapamil and candesartan, and candesartan and/or telmisartan in combination with one or more of anipamil, devapamil, diltiazem, falipamil, felodipine, gallopamil, isradipine, nicardipine, nifedipine, nimodipine, nisoldipine, tiapamil, and verapamil) target stress-induced hormones in different regions of the brain simultaneously. The combinations of agents described herein, targeting both the adrenergic and the brain RAAS simultaneously with positive pluri-network effects, has never been contemplated or studied until now.

Administration to a subject of the compositions and dosage forms of the present invention containing a first agent selected from one or more of anipamil, devapamil, diltiazem, falipamil, felodipine, gallopamil, isradipine, nicardipine, nifedipine, nimodipine, nisoldipine, tiapamil, and verapamil and a second agent selected from one or more of candesartan and telmisartan, delivered simultaneously or sequentially as a combination treatment, is useful for the treatment of brain and/or behavioral disorders and their associated symptoms (i.e., anxiety, apathy, cognitive difficulty, depression, fatigue, headache, insomnia, irritability, body pain, and psychosis. The combination treatments lead to positive psychosocial outcomes which, over time, correlate with significant improvements in overall measures of quality of life. For example, the compositions are associated with achieving patient advances along Maslow's hierarchy of needs, which is comprehensive outcome in healthcare that reflects not only symptomatology but also impact across a broad range of social determinants of health. In some embodiments, therapeutic combinations described herein positively affect the well-being of a subject undergoing treatment (e.g., increasing cognitive function, increasing life satisfaction, increasing meaning and purpose, increasing emotional support, increasing instrumental support, increasing friendship, increasing life satisfaction).

### Pharmacological Effects

Without intending to be bound by theory, the pharmacological effects of compositions comprising a first agent selected from one or more of anipamil, devapamil, diltiazem, falipamil, felodipine, gallopamil, isradipine, nicardipine, nifedipine, nimodipine, nisoldipine, tiapamil, and verapamil and a second agent selected from one or more of candesartan and telmisartan relate to the physiology of adrenalin and brain renin angiotensin aldosterone systems (RAAS). When a human senses danger, two independent body systems change the intrinsic, functional connectivity of the brain.

The adrenergic system, also known as the sympathetic/parasympathetic system, influences functional connectivity via signaling by the neurotransmitter known as norepinephrine (aka “adrenalin”). When norepinephrine is produced in the adrenal gland, it travels to the brain, where it binds to beta receptors. When beta receptors bind norepinephrine, changes occur such as pupillary dilation, reduction of pain sensation, and increased vigilance.

The brain renin angiotensin aldosterone system, also known as the brain RAAS system, influences functional connectivity via the neurotransmitter known as angiotensin. When the angiotensin precursor is produced in the kidneys, it travels through the lung and to the brain, where it binds to angiotensin receptors. When brain angiotensin receptors bind angiotensin, changes occur in the coupling between arterioles and astrocytes, a process known as neurovascular coupling, which causes focal changes in cerebral blood flow.

Not intending to be bound by any particular mode of action, when the adrenalin and brain RAAS systems are activated, the functional connectivity of the brain changes. This change in connectivity is an evolutionarily-preserved stress response system that helps the brain to adapt to metabolic demand. If maintained chronically, this can create an unequilibrated state of functional connectivity which can be detrimental. Several factors can promote chronic disequilibrium, including the built environment, social determinants of health and individual differences. These factors can be especially challenging for patients with a brain and/or behavioral health disorder. Not being bound by theory, compositions comprising a first agent selected from one or more of anipamil, devapamil, diltiazem, falipamil, felodipine, gallopamil, isradipine, nicardipine, nifedipine, nimodipine, nisoldipine, tiapamil, and verapamil and a second agent selected from one or more of candesartan and telmisartan can re-equilibrate the changes in functional connectivity that are associated with a brain and/or behavioral health disorder.

Not wishing to be bound by theory, combination treatments involve administering to a subject compositions comprising a first agent selected from one or more of anipamil, devapamil, diltiazem, falipamil, felodipine, gallopamil, isradipine, nicardipine, nifedipine, nimodipine, nisoldipine, tiapamil, and verapamil and a second agent selected from one or more of candesartan and telmisartan. Such combinations act on central functional networks that are common to more than one neuropsychiatric disorder. Without intending to be bound by theory, the unique combination of an angiotensin II receptor blockade with a concomitant adrenergic blockade leads to re-equilibration of distribution of cerebral blood flow particularly in the diencephalon, which is the region of the brain important for social and emotional awareness. In other words, by influencing functional networks related to cognition (telencephalon) simultaneously with influencing functional networks related to emotion (mesencephalon), functional networks related to social cognition (diencephalon) are equilibrated. This hypothesis is based on findings of conspicuous social changes in patients who took combination therapy. For example, bi-directional changes were observed in social and dressing behavior in patients taking combination treatment. Specifically, introverted patients displayed more extraversion while on combination treatment, whereas extraverted patients displayed more introversion while on combination treatment. The results of a study described in the Examples provided herein showed an improvement in social satisfaction in patients on combination treatment. The hypothesis that combination treatment leads to equilibrating effects in functional brain networks is further supported by analyses of results from the Weber test, performed as part of routine visits, which measures sensorineural function, which is influenced by lateralization of the brain's auditory functional network. In the clinic, patients who initiated combination treatment or changed from monotherapy to combination treatment displayed shifts from one visit to the next in lateralization of the Weber test. Shifts in lateralization of the Weber test do not occur with standard of care medications. Shifts in lateralization of the Weber do not occur when patients take the individual components of combination treatment. Remarkably, these shifts, like the changes in social outcomes, were bi-directional whereby some patients shifted towards the right and other patients shifted towards the left.

Not wishing to be bound by theory, the novel bi-directional outcomes associated with compositions comprising a first agent selected from one or more of anipamil, devapamil, diltiazem, falipamil, felodipine, gallopamil, isradipine, nicardipine, nifedipine, nimodipine, nisoldipine, tiapamil, and verapamil and a second agent selected from one or more of candesartan and telmisartan suggest that combination treatment may serve a regulatory function in brain regions associated with the auditory and/or other functional brain networks. Since changes in auditory function correlated with clinical improvements in social function, therapeutic combinations described herein may affect not only auditory function, but also the interoceptive/exteroceptive awareness as well as the social communication skills that rely on auditory functioning. In fact, the auditory cortex participates directly in networks for emotional processing and communication (Disability and poor quality of life associated with comorbid anxiety disorders and physical conditions. Sareen J, Jacobi F, Cox B J, Belik S L, Clara I, Stein M B. Arch Intern Med. 2006 Oct. 23; 166 (19): 2109-16. doi: 10.1001/archinte.166.19.2109).

### Compositions of the Invention Modulate Adrenergic and Angiotensin Function

Compositions comprising a first agent selected from one or more of anipamil, devapamil, diltiazem, falipamil, felodipine, gallopamil, isradipine, nicardipine, nifedipine, nimodipine, nisoldipine, tiapamil, and verapamil and a second agent selected from one or more of candesartan and telmisartan comprise one ingredient for adrenergic modulation and one ingredient for brain RAAS. No pharmaceutical agents have been designed to target both systems simultaneously. This is a critical treatment gap because both of these systems interact when patients with brain and/or behavioral disorders experience the oft-untreatable symptoms that cause suffering.

### The Adrenalin and Angiotensin Systems Affect Different Functional Brain Networks.

Without intending to be bound by theory, the effect of compositions comprising a first agent selected from one or more of anipamil, devapamil, diltiazem, falipamil, felodipine, gallopamil, isradipine, nicardipine, nifedipine, nimodipine, nisoldipine, tiapamil, and verapamil and a second agent selected from one or more of candesartan and telmisartan depends in part on the relative density of the adrenalin and angiotensin receptors within each one of three phylogenetic levels of the brain. The mesencephalon is the oldest level of the brain, located at the bottom of the brain, just above the spinal cord. The telencephalon is the newest level of the brain to evolve, located at the top of the brain. Between these levels is the diencephalon. Not being bound by theory, each level of the brain contains brain regions rich with receptors that participate in several networks that subserve different neurological processes.

Since beta receptors (which bind norepinephrine) are located more densely within mesencephalon than telencephalon, the effect of norepinephrine is strongest in brain networks that rely on mesencephalic brain regions such as the brainstem. Since angiotensin receptors (which bind angiotensin) are located more densely within telencephalon than mesencephalon, the effect of angiotensin is strongest in brain networks that rely on telencephalic brain regions such as the prefrontal cortex. Clinically, modulating the adrenergic system influences neuropsychiatric symptoms (e.g., insomnia, fatigue, apathy, pain, anxiety) because mesencephalic networks have extensive connections to body organs. Modulating the angiotensin system influences different neuropsychiatric symptoms (e.g., cognitive impairment, headache, aura) because telencephalic networks have extensive connections to brain regions for sensory processing.

Each functional brain network spans different regions of the older and newer brain. The key functional brain networks implicated in brain and/or behavioral health disorders can include the default mode network, the salience, somatosensory, visual, auditory, and limbic networks, among others. Some of these brain networks rely heavily on newer brain regions. For example, the default mode network coordinates among regions mostly located within the telencephalon (the highest phylogenetic level). The salience network, on the other hand, coordinates regions within the mesencephalon (the lowest phylogenetically level).

TABLE 1
Functional Brain Network Regions Organized by Phylogenetic Levels

| | Telencephalon | Diencephalon | Mesencephalon |
|---|---|---|---|
| Salience | Dorsal anterior cingulate/paracingulate cortex | Hypothalamus | Sublenticular |
| Network | Frontoinsular cortices. | Dorsomedial | extended amygdala |
| | Anterior insula | thalamus | Ventral |
| | Putamen | | striatopallidum |
| | Anterior temporal pole | | Periaqueductal gray |
| | | | Substantia |
| | | | nigra/ventral |
| | | | tegmental area |
| Default Mode | Posterior cingulate cortex | Thalamus | Medial mesencephalic |
| Network | Retrosplenial cortex | | regions |
| | VM prefrontal cortex | | |
| | AM prefrontal cortex | | |
| | Dorsal prefrontal cortex | | |
| | Temporal pole | | |
| | Middle temporal gyrus | | |
| | Hippocampus | | |
| | Parahippocampal cortex | | |
| | Amygdala | | |
| | Posterior parietal cortex | | |
| | Basal forebrain | | |
| Somatosensory | Primary somatosensory cortex | | |
| | Primary motor cortex | | |
| | Somatosensory association cortex | | |
| Visual | primary somatosensory cortex | | |
| | primary motor gyrus | | |
| | angular gyrus | | |
| | lateral occipital cortex | | |
| | mid-cingulate cortex | | |
| | anterior cingulate cortex | | |
| | supplementary motor area | | |
| | inferior frontal gyrus | | |
| | precentral gyrus | | |
| | temporal pole | | |
| | precuneous | | |
| Auditory | Bilateral superior temporal gyrus specifically | | |
| Network | Heschl's gyrus and Planum temporale | | |
| Limbic | Hippocampus | Hypothalamus | |
| | | Anterior Thalamic | |
| | | Nuclei | |

### Therapeutic Combinations of the Invention Normalize Whole Brain Functional Connectivity.

In embodiments, compositions comprising a first agent selected from one or more of anipamil, devapamil, diltiazem, falipamil, felodipine, gallopamil, isradipine, nicardipine, nifedipine, nimodipine, nisoldipine, tiapamil, and verapamil and a second agent selected from one or more of candesartan and telmisartan achieve pluri-network effects because equilibration in lower brain networks influences equilibration of higher networks and vice versa. The compositions comprise at least two ingredients in order to block two different stress-related systems (adrenalin system and brain RAAS). Each of these systems can play a key role in shifting functional connectivity of the brain as needed. Not being bound by theory, anipamil, devapamil, diltiazem, falipamil, felodipine, gallopamil, isradipine, nicardipine, nifedipine, nimodipine, nisoldipine, tiapamil, and verapamil and combinations thereof interact with beta receptors within mesencephalon. Not being bound by theory, telmisartan and candesartan interact with angiotensin receptors within telencephalon.

Compositions comprising a first agent selected from one or more of anipamil, devapamil, diltiazem, falipamil, felodipine, gallopamil, isradipine, nicardipine, nifedipine, nimodipine, nisoldipine, tiapamil, and verapamil and a second agent selected from one or more of candesartan and telmisartan, as detailed in the Examples below, have led to clinical effects that suggest a regulation in functional connectivity throughout the brain. The clinical effect of the compositions included normalization not only of emotional, but also social and cognitive symptoms due to the unique pluri-network effects achieved through this synergy. Not being bound by theory, since these functions emanate from three distinct layers (diencephalon, mesencephalon and telencephalon), compositions comprising a first agent selected from one or more of anipamil, devapamil, diltiazem, falipamil, felodipine, gallopamil, isradipine, nicardipine, nifedipine, nimodipine, nisoldipine, tiapamil, and verapamil and a second agent selected from one or more of candesartan and telmisartan alter connectivity at all three levels. The pluri-network effects of the compositions enable patients to make swift psychosocial progress, which further improves whole brain connectivity (Safety Needs Mediate Stressful Events Induced Mental Disorders. Zheng Z, Gu S, Lei Y, Lu S, Wang W, Li Y, Wang F. Neural Plast. 2016; 2016:8058093. doi: 10.1155/2016/8058093. Epub 2016 Sep. 21). Not being bound by theory, improvement in symptomatology referable to all three phylogenetic levels does not occur when either agent is administered alone. Improvement in symptomatology referable to all three phylogenetic levels does not occur with any known standard of care treatment.

Prevention of Brain and/or Behavioral Health Disorders, and Medical Disorders

The pluri-network effects of compositions comprising a first agent selected from one or more of anipamil, devapamil, diltiazem, falipamil, felodipine, gallopamil, isradipine, nicardipine, nifedipine, nimodipine, nisoldipine, tiapamil, and verapamil and a second agent selected from one or more of candesartan and telmisartan discussed herein suggest that treatment with the compositions can treat disorders (e.g., panic disorder) affecting different parts of the brain at different times throughout the life course. Many neuropsychiatry disorders that are amenable to preventative treatment feature disequilibrium in more than one functional brain network, suggesting that these disorders are promising candidates for prophylactic treatment with a combination therapy described herein. For example, chronic pain disorders, which become more refractory to treatment across the life course if unaddressed, feature abnormalities in visual, salience and default mode networks that can be detectable prior to the onset of significant disability. Neurodevelopmental disorders such as ADHD, autism spectrum disorder and learning disorder, which become more refractory to treatment across the life course if unaddressed, feature pluri-network changes. Psychotic disorders such as schizophrenia, which becomes more refractory to treatment across the life course if unaddressed, feature functional abnormalities in multiple brain networks. Anxiety disorders become more refractory to treatment across the life course if unaddressed and show pluri-network effects, with some specific differences according to the disorder. Compositions comprising a first agent selected from one or more of anipamil, devapamil, diltiazem, falipamil, felodipine, gallopamil, isradipine, nicardipine, nifedipine, nimodipine, nisoldipine, tiapamil, and verapamil and a second agent selected from one or more of candesartan and telmisartan are a promising preventative treatment for these conditions because they can improve whole brain functional connectivity despite individual differences in network dis-equilibration among the disorders. No current treatments are available to prevent neuropsychiatric disorders that worsen if left unmitigated across the life course. No research has ever been done regarding use of such compositions for prevention of neuropsychiatric disease or disability.

Compositions comprising a first agent selected from one or more of anipamil, devapamil, diltiazem, falipamil, felodipine, gallopamil, isradipine, nicardipine, nifedipine, nimodipine, nisoldipine, tiapamil, and verapamil and a second agent selected from one or more of candesartan and telmisartan can be used for prevention of neurodegenerative disease risk beginning in midlife. The compositions can cumulatively normalize cerebral metabolism, which can be dysregulated in preclinical neurodegenerative disease states. Different Neurodegenerative disorders such as Alzheimer's disease, Parkinson's disease and frontotemporal lobar degeneration feature disequilibrium in different brain networks that stem from abnormal cerebral metabolism. No current treatments are available that can normalize cerebral metabolism in patients at risk for neurodegenerative diseases. Use of such compositions has never been studied in the context of neurodegenerative disease prevention or treatment.

Since compositions comprising a first agent selected from one or more of anipamil, devapamil, diltiazem, falipamil, felodipine, gallopamil, isradipine, nicardipine, nifedipine, nimodipine, nisoldipine, tiapamil, and verapamil and a second agent selected from one or more of candesartan and telmisartan can normalize whole brain cerebral connectivity, the compositions can be used for certain indications that are outside the boundaries of neuropsychiatry (i.e., cancer, autoimmune, autonomic, among others). Dysregulated functional connectivity can affect risk for medical disorders because brain connectivity affects the health of the immune system. Specifically, medical disorders that conspicuously present around developmental windows (such as age 25, when the brain finishes myelinating) are candidates for a novel, brain-based, preventative treatment including treatment with the compositions.

### Pharmaceutical Compositions

The disclosure provides pharmaceutical compositions comprising an effective amount of a first agent selected from one or more of anipamil, devapamil, diltiazem, falipamil, felodipine, gallopamil, isradipine, nicardipine, nifedipine, nimodipine, nisoldipine, tiapamil, and verapamil and an effective amount of a second agent selected from one or more of candesartan and telmisartan. Such combinations may be co-formulated or formulated separately but administered concurrently or sequentially. When the first and second agents are administered in combination, they are useful for the treatment of brain and/or behavioral health disorders (e.g., panic disorder). In some embodiments, the combination therapy is administered to treat one or more of the ten cardinal neuro-psychiatric symptoms (i.e., anxiety, apathy, cognitive difficulty, depression, fatigue, headache, insomnia, irritability, body pain, and psychosis).

In embodiments, the compositions of the present invention contain a first agent selected from one or more of anipamil, devapamil, diltiazem, falipamil, felodipine, gallopamil, isradipine, nicardipine, nifedipine, nimodipine, nisoldipine, tiapamil, and verapamil and a second agent selected from one or more of candesartan and telmisartan for use in a combination treatment. In embodiments, the first agent is verapamil and the second agent is telmisartan and/or candesartan. In some cases, the first agent is selected to influence the beta receptors in the mesencephalon and the second agent is selected to interact with angiotensin receptors in the telencephalon. The agents can be selected so that the first agent influences the adrenalin system and the second agent influences the brain renin angiotensin aldosterone system. In embodiments, the compositions are associated with modulation of both adrenergic and angiotensin function.

In certain embodiments, the compositions of the present invention can prevent, inhibit, or disrupt, or reduce by at least 10%, 25%, 50%, 75%, or even 100% one or more of anxiety, migraine, depression, cognitive difficulty, anger, apathy, fatigue, body pain, psychosis, and insomnia that manifest in individuals afflicted with a disorder having one or more psychiatric, psychological and/or neurological components.

Pharmaceutically acceptable salts of anipamil, candesartan, devapamil, diltiazem, falipamil, felodipine, gallopamil, isradipine, nicardipine, nifedipine, nimodipine, nisoldipine, telmisartan, tiapamil, and verapamil, or combinations thereof are contemplated herein for the treatment of one or more of the ten cardinal neuropsychiatric symptoms (i.e., anxiety, apathy, cognitive difficulty, depression, fatigue, headache, insomnia, irritability, body pain, and psychosis). The term “pharmaceutically acceptable salt” also refers to a salt prepared by contacting an agent (e.g., anipamil, candesartan, devapamil, diltiazem, falipamil, felodipine, gallopamil, isradipine, nicardipine, nifedipine, nimodipine, nisoldipine, telmisartan, tiapamil, or verapamil), where the agent has an acidic functional group (e.g., a carboxylic acid functional group), with a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)-amine, 2-hydroxy-tert-butylamine, or tris-(hydroxymethyl)methylamine, N, N,-di-lower alkyl-N-(hydroxy lower alkyl)-amines, such as N,N-dimethyl-N-(2-hydroxyethyl)-amine, or tri-(2-hydroxyethyl)amine; N-methyl-D-glucamine; and amino acids such as arginine, lysine, and the like. The term “pharmaceutically acceptable salt” also refers to a salt prepared by contacting an agent (e.g., anipamil, candesartan, devapamil, diltiazem, falipamil, felodipine, gallopamil, isradipine, nicardipine, nifedipine, nimodipine, nisoldipine, telmisartan, tiapamil, or verapamil), where the agent has a basic functional group (e.g., an amino functional group), with a pharmaceutically acceptable inorganic or organic acid. Suitable acids include, but are not limited to, hydrogen sulfate, citric acid, acetic acid, oxalic acid, hydrochloric acid, hydrogen bromide, hydrogen iodide, nitric acid, phosphoric acid, isonicotinic acid, lactic acid, salicylic acid, tartaric acid, ascorbic acid, succinic acid, maleic acid, besylic acid, fumaric acid, gluconic acid, glucaronic acid, saccharic acid, formic acid, benzoic acid, glutamic acid, methanesulfonic acid, ethanesulfonic acid, benzenesulfonic acid, and p-toluenesulfonic acid.

The compositions of the present invention can contain a salt of magnesium, optionally magnesium oxide (MgO).

### Pharmaceutical Therapeutics

For therapeutic uses, compositions comprising a first agent selected from one or more of anipamil, devapamil, diltiazem, falipamil, felodipine, gallopamil, isradipine, nicardipine, nifedipine, nimodipine, nisoldipine, tiapamil, and verapamil, and a second agent selected from one or more of candesartan and telmisartan can be administered systemically. Preferable routes of administration include, for example, oral administration or subcutaneous, intravenous, interperitoneally, intramuscular, or intradermal injections that provide continuous, sustained levels of the drug in the patient. Treatment of human patients or other animals will be carried out using a therapeutically effective amount (e.g., for treatment of a panic disorder) of a therapeutic identified herein in a physiologically-acceptable carrier. Anipamil, devapamil, diltiazem, falipamil, felodipine, gallopamil, isradipine, nicardipine, nifedipine, nimodipine, nisoldipine, tiapamil, and/or verapamil in combination with candesartan and/or telmisartan can be formulated in a pharmaceutically-acceptable buffer such as physiological saline. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of a brain and/or behavioral health disorder. The clinical symptoms, in some embodiments, are the ten cardinal neuropsychiatric symptoms (i.e., anxiety, apathy, cognitive difficulty, depression, fatigue, headache, insomnia, irritability, pain (e.g., body pain), and psychosis). Generally, amounts will be in the range of those used for other agents used in the treatment of a brain and/or behavioral health disorder. In some embodiments, a composition comprising verapamil and telmisartan, verapamil and candesartan, or candesartan and/or telmisartan in combination with one or more of anipamil, devapamil, diltiazem, falipamil, felodipine, gallopamil, isradipine, nicardipine, nifedipine, nimodipine, nisoldipine, tiapamil, and verapamil is administered at a dosage that is effective at reducing one or more of the ten cardinal neuropsychiatric symptoms (i.e., anxiety, apathy, cognitive difficulty, depression, fatigue, headache, insomnia, irritability, body pain, and psychosis. Effectiveness of the administration can be determined by a method known to one skilled in the art, or using any assay that measures one or more of the ten neuropsychiatric symptoms (e.g., behavioral assessment, neuropsychological testing, etc.). For example, in some presently disclosed embodiments, effectiveness of the treatment can be measured by patient self-reports of symptoms and/or via NIH toolbox testing. Effectiveness can also be measured outcomes on disease-specific, gold-standard outcome measures, including measures that assess not only symptoms and function but also overall life satisfaction or quality of life.

### Formulation of Pharmaceutical Compositions

The administration of a composition containing a first agent selected from one or more of anipamil, devapamil, diltiazem, falipamil, felodipine, gallopamil, isradipine, nicardipine, nifedipine, nimodipine, nisoldipine, tiapamil, and verapamil and a second agent selected from one or more of candesartan and telmisartan can be administered to a subject for the treatment of a brain and/or behavioral health disorder (e.g., panic disorder) by any suitable means that results in a concentration of the therapeutic that, combined with other components, is effective in ameliorating, reducing, or stabilizing one or more of the ten cardinal neuropsychiatric symptoms. In some embodiments, the combination treats one or more of the ten cardinal neuropsychiatric symptoms (i.e., anxiety, apathy, cognitive difficulty, depression, fatigue, headache, insomnia, irritability, body pain, and psychosis). The compositions can contain a salt of magnesium, optionally magnesium oxide (MgO). The composition can be contained in any appropriate amount any suitable carrier substance, that is generally present in an amount of 1-95% by weight of the total weight of the composition. The composition can be provided in a dosage form that is suitable for oral administration. In some embodiments, the composition can be provided in a dosage form that is suitable for a parenteral (e.g., subcutaneously, intravenously, intramuscularly, or intraperitoneally) administration route. The pharmaceutical compositions can be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Human dosage amounts can initially be determined by extrapolating from the amount of anipamil, candesartan, devapamil, diltiazem, falipamil, felodipine, gallopamil, isradipine, nicardipine, nifedipine, nimodipine, nisoldipine, telmisartan, tiapamil, or verapamil, or combinations thereof, used in mice. Dosages can also be determined based on dosages for the effective treatment of disorders for which the individual agents have been indicated in humans. The dosage (optionally a daily dosage) amount of one or more of anipamil, candesartan, devapamil, diltiazem, falipamil, felodipine, gallopamil, isradipine, nicardipine, nifedipine, nimodipine, nisoldipine, telmisartan, tiapamil, and verapamil, individually or collectively, can be from about 1 mg to about 720 mg, 120 mg to about 480 mg, from about 250 mg to about 360 mg, from about 200 mg to about 500 mg, from about 250 mg to about 350 mg, from about 120 mg to about 720 mg. The dosage amount of the first agent can be from about 20 mg to about 720 mg, from about 30 mg to about 120 mg, from about 50 mg to about 200 mg, from about 60 mg to about 360 mg, from about 200 mg to about 500 mg, or about 288 mg, about 15 mg, or about 30 mg. The dosage amount of the second agent can be from about 1 mg to about 240 mg, from about 2.5 mg to about 40 mg, from about 20 mg to about 180 mg, from about 80 mg to about 400 mg, from about 45 mg to about 180 mg, from about 80 mg to about 320 mg, from about 45 mg to about 150 mg, from about 90 mg to about 200 mg, or about 96 mg, about 2.5 mg, or about 5 mg. In embodiments, the dosage amount of the second agent is greater than 70 mg, 75 mg, 80 mg, 85 mg, 90 mg, 95 mg, 100 mg, or 150 mg. The dosage of the first agent or components thereof and the second agent or components thereof can be contained in one or multiple dosage forms. The dosage amounts of the first agent and the second agent may vary between adult formulations and pediatric formulations.

In some embodiments, the daily dosage of either the first or the second agent is administered more than once per day. For example, in some embodiments, the daily administration (e.g., 80 mg) is delivered in two 40 mg doses twice per day.

The first agent and the second agent can be administered to a subject (optionally as a dosage form) at a dosage ratio (mass:mass) of the first agent (e.g., anipamil, devapamil, diltiazem, falipamil, felodipine, gallopamil, isradipine, nicardipine, nifedipine, nimodipine, nisoldipine, tiapamil, and verapamil, or combinations thereof) to the second agent (e.g., candesartan, telmisartan, or combinations thereof) of about or at least about 1:1, 2:1, 3:1, 4:1, 5:1, 6:1, 7:1, or 8:1. In some embodiments, the dosage ratio of the first agent (e.g., anipamil, devapamil, diltiazem, falipamil, felodipine, gallopamil, isradipine, nicardipine, nifedipine, nimodipine, nisoldipine, tiapamil, and verapamil, or combinations thereof) to the second agent (e.g., candesartan, telmisartan, or combinations thereof) is less than about 3:1, 4:1, 5:1, 6:1, 7:1, or 8:1. The first agent (e.g., anipamil, devapamil, diltiazem, falipamil, felodipine, gallopamil, isradipine, nicardipine, nifedipine, nimodipine, nisoldipine, tiapamil, and verapamil, or combinations thereof) can be administered to a subject at a daily dosage that is about double or triple a daily dosage of the second agent (e.g., candesartan, telmisartan, or combinations thereof) administered to the subject. In some embodiments, the dosage amounts for the first agent (e.g., anipamil, devapamil, diltiazem, falipamil, felodipine, gallopamil, isradipine, nicardipine, nifedipine, nimodipine, nisoldipine, tiapamil, and verapamil, or combinations thereof), the second agent (e.g., candesartan, telmisartan, or combinations thereof), or components thereof (e.g., anipamil, candesartan, devapamil, diltiazem, falipamil, felodipine, gallopamil, isradipine, nicardipine, nifedipine, nimodipine, nisoldipine, telmisartan, tiapamil, and verapamil, or combinations thereof), individually or in combination, can vary from between about 0.1 mg compound/Kg body weight to about 2 mg compound/Kg body weight; or from about 0.5 mg/Kg body weight to about 2 mg/Kg body weight or from about 1.0 mg/Kg body weight to about 2 mg/Kg body weight; or from about 1.5 mg/Kg body weight to about 2 mg/Kg body weight; or from about 0.1 mg/Kg body weight to about 1.5 mg/Kg body weight; or from about 0.10 mg/Kg body weight to about 1.0 mg/Kg body weight; or from about 0.10 mg/Kg body weight to about 0.5 mg/Kg body weight. In other embodiments this dose can be about 0.1, 0.25, 0.5, 0.75, 1.0, 1.25, 1.5, 1.75, 2.0, 2.25, 2.5, 2.75, or 3.0 mg/Kg body weight. In other embodiments, it is envisaged that doses can be in the range of about 0.2 mg compound/Kg body to about 2 mg compound/Kg body. Of course, the dosage amounts can be adjusted upward or downward, as is routinely done in such treatment protocols, depending on the results of the initial clinical trials and the needs of a particular patient.

The dosage amount (optionally a daily dosage) of a magnesium salt (e.g., MgO) in embodiments is about or at least about 100 mg, 150 mg, 200 mg, 250 mg, 300 mg, 350 mg, 400 mg, or 500 mg. The dosage amount (optionally a daily dosage) of a magnesium salt (e.g., MgO) in embodiments is no more than about 250 mg, 300 mg, 350 mg, 400 mg, 450 mg, or 500 mg.

Pharmaceutical compositions according to the presently disclosed embodiments can be formulated to release the active compound (e.g., anipamil, candesartan, devapamil, diltiazem, falipamil, felodipine, gallopamil, isradipine, nicardipine, nifedipine, nimodipine, nisoldipine, telmisartan, tiapamil, and verapamil, or combinations thereof) substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition near intended targeted cells (e.g., brain cells); (v) formulations that allow for convenient dosing, such that doses are administered, for example, orally once or twice per day; and (vi) formulations that target calcium channels and angiotensin receptors by using carriers or chemical derivatives to deliver the therapeutic agent to a particular cell type (e.g., brain cell). For some applications, controlled release formulations obviate the need for frequent dosing during the day to sustain the plasma level at a therapeutic level.

Immediate release formulations of the compositions of this disclosure may be formulated to release the active compound (e.g., anipamil, candesartan, devapamil, diltiazem, falipamil, felodipine, gallopamil, isradipine, nicardipine, nifedipine, nimodipine, nisoldipine, telmisartan, tiapamil, and verapamil, or combinations thereof) substantially immediately under one or more of: i) fasted gastric conditions; ii) fed gastric conditions; or iii) intestinal conditions. Such administration conditions may include a wide range of associated pH conditions, such as pH 1 or less, pH 4-6, pH 5-8, or greater than pH 8. Substantially immediate release may include, but is not limited to, 80% or greater release within 1 hour of administration, 90% or greater release within 1 hour of administration, 90% or greater release within 2 hours of administration, 90% or greater release within 1.5 hours of administration, 95% or greater release within 2 hours of administration, 95% or greater release within 1.5 hours of administration.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Solid Dosage Forms for Oral Use

Formulations for oral use include tablets containing the active ingredients (e.g., one or more of anipamil, candesartan, devapamil, diltiazem, falipamil, felodipine, gallopamil, isradipine, nicardipine, nifedipine, nimodipine, nisoldipine, telmisartan, tiapamil, and verapamil) in a mixture with non-toxic pharmaceutically acceptable excipients. Such formulations are known to the skilled artisan. Excipients can be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets are uncoated in some embodiments and coated in other embodiments. The tablets can be coated by known techniques, optionally to delay disintegration and absorption in the gastrointestinal tract and thereby provide a sustained action over a longer period. The coating can be adapted to release the active drug or drugs in a predetermined pattern (e.g., in order to achieve a controlled release formulation) or adapted not to release the active drug until after passage of the stomach (enteric coating). The coating, in some embodiments, is a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate can be employed.

The solid tablet compositions include, in some embodiments, a coating adapted to protect the composition from unwanted chemical changes, (e.g., chemical degradation prior to the release of the first and/or second agent). In some embodiments, the coating is applied on the solid dosage form in a similar manner as that described in Encyclopedia of Pharmaceutical Technology, supra.

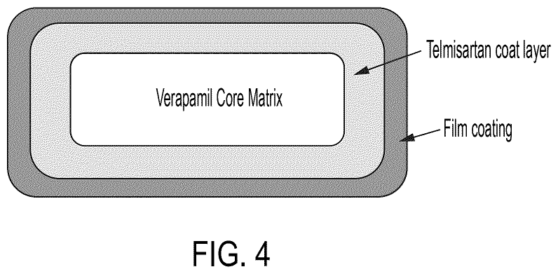

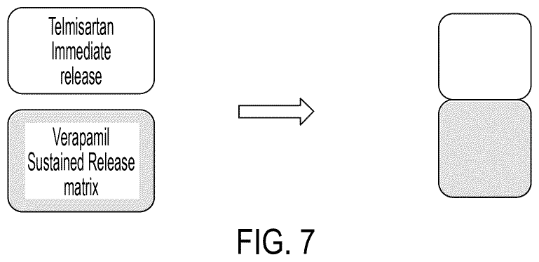

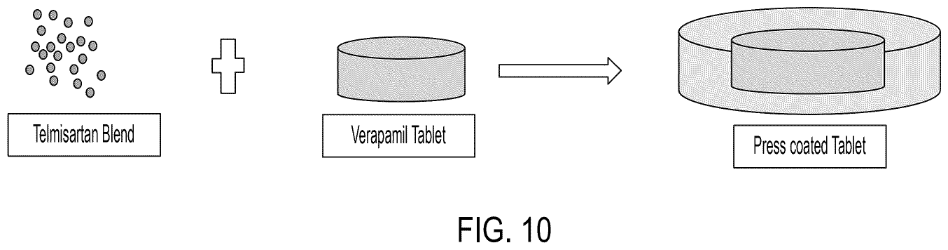

One or more of anipamil, candesartan, devapamil, diltiazem, falipamil, felodipine, gallopamil, isradipine, nicardipine, nifedipine, nimodipine, nisoldipine, telmisartan, tiapamil, and verapamil are mixed together in the tablet or partitioned. In one example, a first agent (e.g. one or more of anipamil, devapamil, diltiazem, falipamil, felodipine, gallopamil, isradipine, nicardipine, nifedipine, nimodipine, nisoldipine, tiapamil, and verapamil) is contained on the inside of the tablet, and a second agent (e.g., one or more of candesartan and telmisartan) is on the outside, such that a substantial portion of the second agent is released prior to the release of the first agent, for example, such as in FIG. 4. In some embodiments, the second agent (e.g., one or more of candesartan and telmisartan) is contained on the inside of the tablet and the first agent (e.g. one or more of anipamil, devapamil, diltiazem, falipamil, felodipine, gallopamil, isradipine, nicardipine, nifedipine, nimodipine, nisoldipine, tiapamil, and verapamil) is on the outside. In another example, the tablet may be partitioned such that the first agent is contained in a layer which is distinct from the second agent, such as in FIG. 7. In some embodiments, an outer layer of the tablet containing one of the first agent or the second agent is coated onto an inner layer of the tablet containing the other agent, such as in FIG. 4, although the outer layer may be press coated from powder, pellet, particle, or granulate form onto the inner layer, such as in FIG. 10. In some embodiments, the agent contained or coated on the outside of the tablet is formulated in immediate release form and the agent contained in the inside of the tablet is formulated in controlled release form.

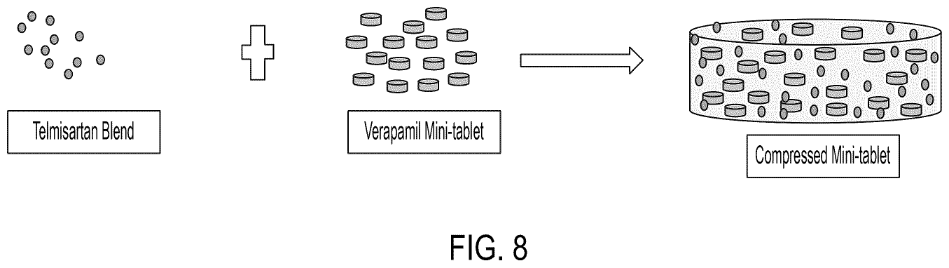

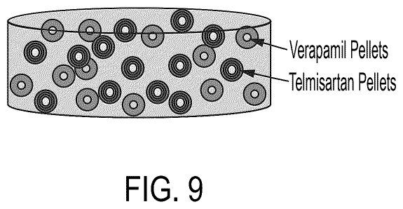

Formulations for oral use also include compressed tablets, wherein the active ingredients (i.e., anipamil, devapamil, diltiazem, falipamil, felodipine, gallopamil, isradipine, nicardipine, nifedipine, nimodipine, nisoldipine, tiapamil, and verapamil, or combinations thereof) are compressed together, such as in FIGS. 8 and 9. In one example, a first agent (e.g. one or more of anipamil, devapamil, diltiazem, falipamil, felodipine, gallopamil, isradipine, nicardipine, nifedipine, nimodipine, nisoldipine, tiapamil, and verapamil) is provided in tablet or mini-tablet form, and a second agent (e.g., one or more of candesartan and telmisartan) is provided in powder, pellet, particle, or granulate form, after which the first and second agents are compressed together to form a compressed tablet. In other embodiments, both agents may instead be provided in powder, pellet, particle, or granulate form, or both agents may be provided in tablet or mini-tablet form prior to compression. In preferred embodiments of these compressed tablets, the second agent is provided in immediate release form.

Formulations for oral use also include push-pull osmotic pump tablets, wherein the active ingredients (i.e., anipamil, devapamil, diltiazem, falipamil, felodipine, gallopamil, isradipine, nicardipine, nifedipine, nimodipine, nisoldipine, tiapamil, and verapamil, or combinations thereof) are contained either on an outer layer of the tablet or within an osmotic core of the tablet, surrounded by a semi-permeable membrane having one or more pores for release of the active ingredients in the osmotic core. In one example, a first agent (e.g. one or more of anipamil, devapamil, diltiazem, falipamil, felodipine, gallopamil, isradipine, nicardipine, nifedipine, nimodipine, nisoldipine, tiapamil, and verapamil) is layered in the osmotic core with a polymeric osmogen layer, both of which are surrounded by a semi-permeable membrane with one or more pores, and a second agent (e.g., one or more of candesartan and telmisartan) is coated on the outside of the semi-permeable membrane. Upon administration, in this example, the second agent is immediately released, whereas the first agent is subject to controlled release as the semi-permeable membrane allows water to pass into the osmotic core which leaches the first agent out through the pores in the membrane, such as through osmotic pressure, and/or pushes the first agent through the pores in the membrane as the polymeric osmogen expands upon absorbing water. In preferred embodiments of these compressed tablets, the second agent is provided in immediate release form.

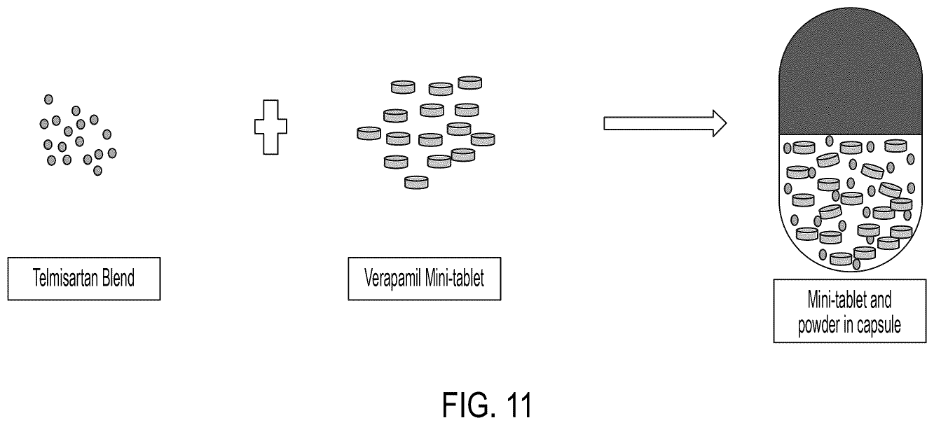

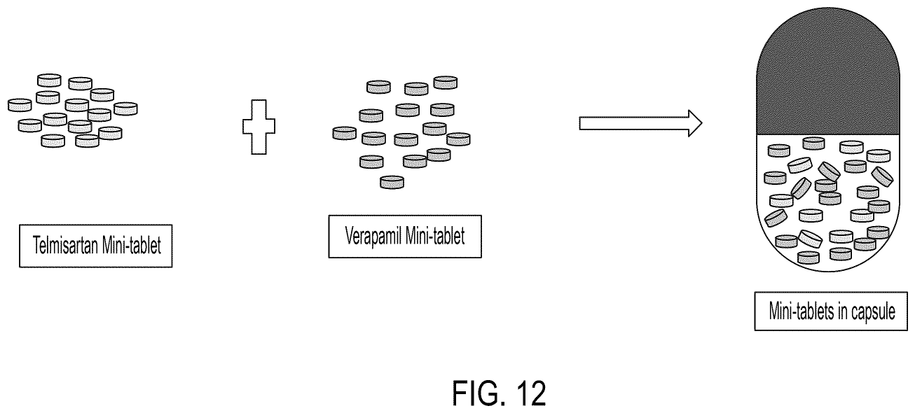

Formulations for oral use also include chewable tablets or capsules (such as hard gelatin capsules), wherein the active ingredients (i.e., anipamil, devapamil, diltiazem, falipamil, felodipine, gallopamil, isradipine, nicardipine, nifedipine, nimodipine, nisoldipine, tiapamil, and verapamil, or combinations thereof) are mixed with an inert solid diluent (e.g., potato starch, lactose, microcrystalline cellulose, calcium carbonate, calcium phosphate, or kaolin), or as soft gelatin capsules, wherein the active ingredients are mixed with water or an oil medium, for example, peanut oil, liquid paraffin, or olive oil, such as in FIGS. 11 and 12. In one example, a first agent (e.g. one or more of anipamil, devapamil, diltiazem, falipamil, felodipine, gallopamil, isradipine, nicardipine, nifedipine, nimodipine, nisoldipine, tiapamil, and verapamil) is provided in tablet or mini-tablet form, and a second agent (e.g., one or more of candesartan and telmisartan) is provided in powder, pellet, particle, or granulate form, after which the first and second agents are encapsulated to form a capsule. In other embodiments, both agents may instead be provided in powder, pellet, particle, or granulate form, or both agents may be provided in tablet or mini-tablet form prior to encapsulation. In preferred embodiments of these capsules, the second agent is provided in immediate release form.

Powders, pellets, particles, and granulates are prepared in some embodiments using the ingredients mentioned above under tablets and capsules in a conventional manner using, e.g., a mixer, a fluid bed apparatus, or a spray drying equipment.

### Controlled and Immediate Release Oral Dosage Forms

Controlled release compositions of a first agent selected from one or more of anipamil, devapamil, diltiazem, falipamil, felodipine, gallopamil, isradipine, nicardipine, nifedipine, nimodipine, nisoldipine, tiapamil, and verapamil and a second agent that is selected from one or more of candesartan and telmisartan (e.g., for oral use) can be constructed to release the agents by controlling the dissolution and/or the diffusion of the active substance. For example, an immediate release formulation is commercially available for verapamil and telmisartan, as are extended release versions of verapamil. The verapamil extended release (ER) formulations release the drug over 12 or 24 hours. Dissolution or diffusion-controlled release can be achieved by appropriate coating of a tablet, capsule, pellet, or granulate formulation of compounds, or by incorporating a composition comprising the first and/or second agents into an appropriate matrix or matrices. A controlled release coating includes, in some embodiments, one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material can also include, e.g., hydrated metylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A composition containing two agents described herein (e.g., one or more of anipamil, devapamil, diltiazem, falipamil, felodipine, gallopamil, isradipine, nicardipine, nifedipine, nimodipine, nisoldipine, tiapamil, and verapamil, and one or more of telmisartan and candesartan) is, in some embodiments, in the form of a buoyant tablet or capsule (i.e., a tablet or capsule that, upon oral administration, floats on top of the gastric content for a certain period of time). A buoyant tablet formulation of the composition can be prepared by granulating a mixture of one or more of the agents, or components thereof, with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Further Combination Therapies

Optionally, the therapeutic combinations of the present disclosure can be administered together with any other standard anti-anxiety, anti-migraine, anti-depression, anti-cognitive difficulty, anti-anger, anti-apathy, anti-fatigue, anti-pain, anti-psychosis, or anti-insomnia therapy such as cognitive behavioral therapy, sedatives, etc.; such methods are known to the skilled artisan and described in Remington's Pharmaceutical Sciences by E. W. Martin. If desired, the therapeutic combinations are administered in combination with any conventional anti-anxiety therapy, including but not limited to, anxiolytic and/or sedative drugs, antipsychotics, mood stabilizers, anticonvulsants, antihistamines, and antidepressants.

The therapeutic combinations or compositions of the present invention can be administered to a subject to treat a neuropsychological condition. The neuropsychological condition can be a brain and/or behavioral disorder. Non-limiting examples of brain and behavioral health disorders include affective disorders, anxiety disorders, neurodegenerative disorders, neurodevelopmental disorders, psychotic disorders, personality disorders, migraine-related disorders, stress-related disorders, somatoform disorders, substance use disorders, seizure disorders, neuroimmune disorders and cerebrovascular disorders. Examples of affective disorders include bipolar disorder, cyclothymia, dysthymia, major depressive disorder, postpartum depression, and seasonal affective disorder. Examples of anxiety disorders include generalized anxiety disorder, panic disorder, social anxiety disorder, post-traumatic stress disorder, obsessive compulsive disorder and specific phobia. Examples of neurodegenerative disorders include Alzheimer's disease, Parkinson's disease, and Dementia with Lewy Bodies. Examples of neurodevelopmental disorders include autism spectrum disorder, attention deficit hyperactive disorder (ADHD) and learning disorders. Examples of psychotic disorders include schizophrenia, schizoaffective disorder and major depression with psychosis. Examples of personality disorders include paranoid, schizoid, schizotypal, antisocial, borderline, histrionic, narcissistic, avoidant, dependent and obsessive-compulsive personality disorder. Examples of migraine-related disorders include migraine with aura, migraine without aura, acephalgic migraine, and vestibular migraine. Examples of stress-related disorders include acute stress reaction, reactive attachment disorder, adjustment disorders, and hyperhidrosis. Examples of somatoform disorders include somatization disorder, hypochondriasis, conversion disorder, body dysmorphic disorder and chronic pain. Examples of substance use disorders include alcohol use disorder and opioid use disorder. Examples of seizure disorders include epileptic disorders and psychogenic non-epileptic seizure disorders. Examples of neuroimmune disorders include multiple sclerosis. Examples of cerebrovascular disorders include cerebrovascular disease, vascular dementia and cerebrovascular accident. The symptoms associated with the disease can be selected from one or more of the “ten cardinal symptoms” associated with brain and behavioral health disorders: anxiety, apathy, cognitive difficulty, depression, fatigue, headache, insomnia, irritability, body pain, and psychosis. Administration of a therapeutic combination, composition, or dosage form of the present invention to a subject can reduce or ameliorate one or more of the symptoms associated with the bran and/or behavioral disorder.

The compositions or therapeutic combinations of the present invention can be administered to a subject in an amount sufficient to alter regional cerebral blood flow in the subject. The cerebral regions can include one or more of the telencephalon, the diencephalon, and the mesencephalon. The compositions or therapeutic combinations of the present invention can be administered to the subject in an amount sufficient to result in hemodynamic equilibrium in functional brain networks that coordinate among regions within the telencephalon, the diencephalon, and the mesencephalon in a subject.

### Selection of Patients for Treatment

The present disclosure provides for the selection of patients who are likely to benefit from treatment with a therapeutic combination described herein. Such patients are selected as having a brain or behavior health disorders or a symptom thereof (e.g., anxiety, apathy, cognitive difficulty, depression, fatigue, headache, insomnia, irritability, body pain, and psychosis). Patients having a brain or behavior health disorders or a symptom thereof are selected for therapy with a combination therapeutic including a first agent selected from one or more of anipamil, devapamil, diltiazem, falipamil, felodipine, gallopamil, isradipine, nicardipine, nifedipine, nimodipine, nisoldipine, tiapamil, and verapamil and a second agent from one or more of telmisartan and candesartan.

Not being bound by theory, patients with the greatest energy supply/demand mismatch can have the greatest benefit from the compositions of the present disclosure. Patients with energy supply/demand mismatch can show the largest changes in functional connectivity before and after combination treatment. Four energy demand/supply phenotypes emerge when one considers energy as binary (high vs. low). The four types are: high demand/high supply (low risk of neuropsychiatric symptomatology), high demand/low supply (high risk of neuropsychiatric symptomatology), low demand/high supply (low risk of neuropsychiatric symptomatology), and low demand/low supply (low risk of neuropsychiatric symptomatology).

### Kits or Pharmaceutical Systems

The present compositions can be assembled into kits or pharmaceutical systems for treating a brain or behavior health disorder or a symptom thereof (e.g., anxiety, migraine, depression, cognitive difficulty, anger, apathy, fatigue, pain, psychosis, or insomnia). Kits or pharmaceutical systems comprise a carrier means, such as a box, carton, tube or the like, having in close confinement therein one or more container means, such as vials, tubes, ampoules, bottles and the like. The kits or pharmaceutical systems can also comprise associated instructions for using the agents of the presently disclosed embodiments. In some embodiments, kits include compositions including one of the first agents (e.g., anipamil, devapamil, diltiazem, falipamil, felodipine, gallopamil, isradipine, nicardipine, nifedipine, nimodipine, nisoldipine, tiapamil, or verapamil) with one of the second agents (e.g., candesartan or telmisartan).

The practice of the presently disclosed embodiments employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, can be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Formulation Compositions

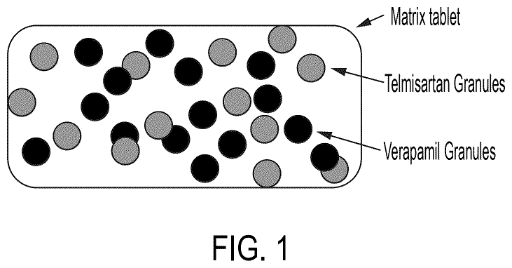

The original product design was a continuous 24-hour release of both telmisartan and verapamil. As such, the original product design included telmisartan and verapamil granulated, followed by a blending step with controlled release polymers and diluents, then subsequently compressed into a tablet. A pictorial presentation is presented in FIG. 1. The formulation composition of batch 341-86 is presented in Table 1 below.

TABLE 1
Formulation Composition of 341-86 Core Matrix Tablet

| | | |
|---|---|---|
| Ingredient | % w/w | mg/unit |

Granulation Stage

| | | |
|---|---|---|
| Verapamil | 36.00 | 288.00 |
| Telmisartan | 12.00 | 96.00 |
| Hydroxypropyl Methylcellulose Grade E5 | 0.63 | 5.00 |
| Purified Water | — | q.s. |
| Total | NA | 389.00 |

Blending stage

| | | |
|---|---|---|
| Hydroxypropyl Methylcellulose Grade K100M | 6.67 | 53.33 |
| Hydroxypropyl Methylcellulose Grade K15M | 13.33 | 106.67 |
| Microcrystalline Cellulose (Avcel PH200) | 14.19 | 113.50 |
| Lactose (Supertab 22AN) | 14.19 | 113.50 |
| Colloidal Silicon Dioxide | 2.00 | 16.00 |
| Magnesium Stearate | 1.00 | 8.00 |
| Total | 100.00 | 800.00 |

Multi-media dissolution was performed. Conditions tested were:
0.1N HCL, which closely simulates fasted gastric conditions
pH 4.5, closely simulating fed gastric conditions
pH 6.8, which closely simulates intestinal conditions

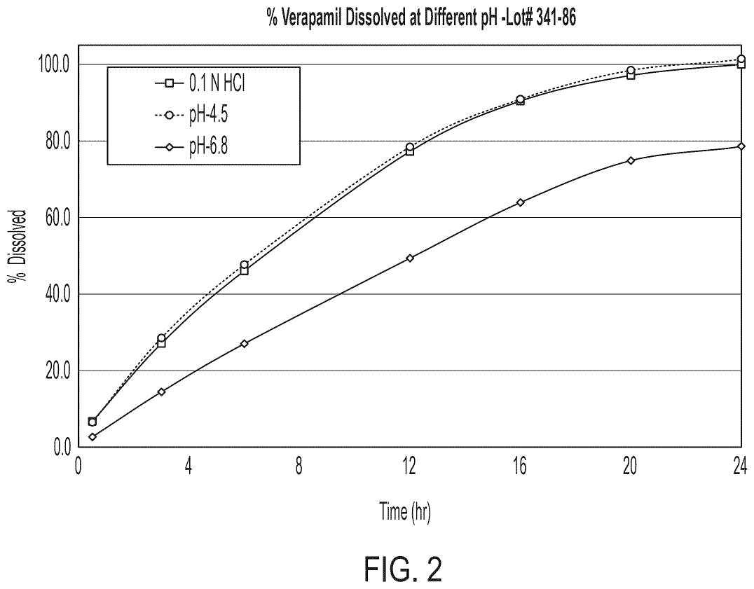

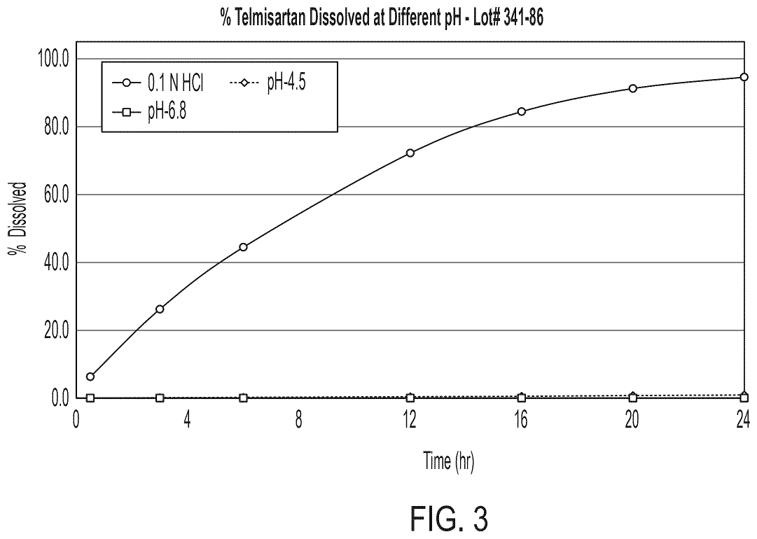

The data is presented in FIG. 2 and FIG. 3. The dissolution testing on batch 341-86 presented unexpected results. While verapamil released steadily over a 24 hour period in all media conditions (FIG. 2), telmisartan had little to no release in pH 4.5 and 6.8 media (FIG. 3), which is the pH associated with normal gastric conditions. This malabsorption presented a challenge to appropriately formulating the combination therapeutic.

### Example 2: Telmisartan/Verapamil Formulation

The potential for lack of absorption in the later gastrointestinal tract due to lack of dissolution of telmisartan was surprising. The design criterion was altered to overcome this challenge. The re-design was a product in which telmisartan releases immediately in gastric pH conditions while verapamil is intended to release over a 24-hour timeframe in all pH conditions. This was achieved by removing telmisartan from the core tablet and adding an immediate release coating of telmisartan on the outside of the verapamil-controlled release matrix tablet. A pictorial representation of the formulation is presented in FIG. 4. Batch 341-109AO was formulated in this manner. The formulation composition details for biphasic drug delivery system are presented in Table 2 below.

TABLE 2
Formulation Composition of 341-109AO

| | | |
|---|---|---|
| Ingredient | % w/w | mg/unit |

Granulation Stage

| | | |
|---|---|---|
| Verapamil Hydrochloride | 29.28 | 288.00 |
| Hydroxypropyl Methylcellulose | 0.51 | 5.00 |
| Purified Water | — | 29.30 |
| Total | 29.78 | 293.00 |

Blending Stage

| | | |
|---|---|---|
| Bulk Verapamil Granules | 29.78 | 293.00 |
| Hydroxypropyl Methylcellulose | 5.42 | 53.33 |
| Hydroxypropyl Methylcellulose | 10.84 | 106.67 |
| Microcrystalline Cellulose PH 200 | 11.54 | 113.50 |
| Lactose Anhydrous | 11.54 | 113.50 |
| Colloidal Silicon Dioxide | 1.63 | 16.00 |
| Magnesium Stearate | 0.81 | 8.00 |
| Total | 71.56 | 704.00 |

Drug Coating Stage

| | | |
|---|---|---|
| Verapamil Tablets | 71.56 | 704.00 |
| Telmisartan | 9.76 | 96.00 |
| Sodium Hydroxide | 6.01 | 59.08 |
| Opadry Clear YS-1-19025-A | 9.76 | 96.00 |
| Purified Water | — | 2259.69 |
| Total | 97.09 | 955.08 |

Aesthetic Coating Stage

| | | |
|---|---|---|
| Verapamil and Telmisartan Tablets | 97.09 | 955.08 |
| Opadry White YS-1-7003 | 2.91 | 28.65 |
| Purified Water | — | 257.87 |
| Total | 100.00 | 983.73 |

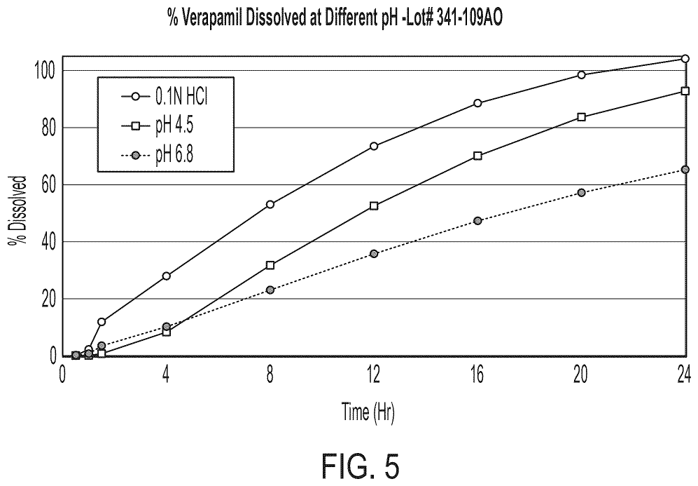

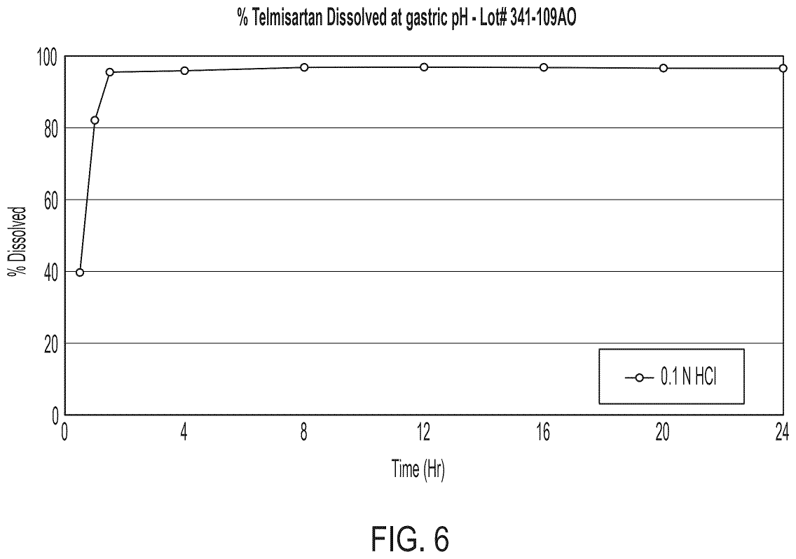

Multi-media dissolution was performed for the 341-109AO and tested for verapamil release. Conditions tested were:
0.1N HCL, which closely simulates fasted gastric conditions.
pH 4.5, closely simulating fed gastric conditions.
pH 6.8, which closely simulates intestinal conditions. The dissolution is presented in FIG. 5. Telmisartan was only tested in gastric pH conditions. The data is presented in FIG. 6.

### OTHER EMBODIMENTS

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference. The invention may be related to PCT International Application No. PCT/US2021/027641, or U.S. application Ser. Nos. 17/477,415, 17/477,444, or 17/996,413, the entirety of each of which are incorporated herein by reference for all purposes.

What is claimed is:

1. A formulation comprising a first composition comprising an effective amount of a first agent that has calcium channel blocking activity, and a second composition comprising an effective amount of a second agent that has angiotensin II receptor blocking activity, wherein the first composition is formulated for extended release of the first agent when administered to a subject and the second composition is formulated for immediate release of the second agent when administered to the subject.

2. The formulation of claim 1, wherein the second composition is formulated to have a faster rate of release of the second agent compared to the rate of release of the first agent for the first composition when administered to a subject.

3. The formulation of claim 1, wherein the second composition is formulated for immediate release and dissolution of the second agent when in contact with a solution having a pH between 1-5.

4. The formulation of claim 1, wherein the second composition is formulated for immediate release and dissolution of the second agent under fed gastric conditions or intestinal conditions in a subject when administered to the subject.

5. A tablet comprising the therapeutic formulation of claim 1.

6. The formulation of claim 1, wherein the first agent is selected from the group consisting of anipamil, devapamil, diltiazem, falipamil, felodipine, gallopamil, isradipine, nicardipine, nifedipine, nimodipine, nisoldipine, tiapamil, and verapamil.

7. The formulation of claim 1, wherein the second agent is telmisartan or candesartan.

8. The formulation of claim 1, wherein the combination is labeled for the treatment of a brain and/or behavioral health disorder or is labeled for the treatment of a symptom of a brain and/or behavioral health disorder selected from the group consisting of anxiety, apathy, cognitive difficulty, depression, fatigue, headache, insomnia, irritability, pain and psychosis.

9. The formulation of claim 1, wherein the first agent is verapamil.

10. The formulation of claim 1, wherein the second agent is telmisartan.

11. The formulation of claim 1, wherein the second composition is formulated such that greater than 80% of the second agent is released within 1 hour of administration to a subject.

12. The formulation of claim 1, wherein the mass ratio of the first agent to the second agent is from about 2:1 to about 5:1.

13. The formulation of claim 1, wherein the amount of first agent is from about 1 to about 4 times the amount of the second agent.

14. The formulation of claim 1, wherein the formulation further comprises magnesium oxide.

15. The formulation of claim 1, wherein the second composition further comprises sodium hydroxide.

16. The tablet of claim 5, wherein the tablet is a compressed tablet or press coated tablet.

17. A pharmaceutical composition comprising the formulation of claim 1 and a pharmaceutically acceptable excipient.

18. A push-pull osmotic pump tablet comprising the formulation of claim 1, wherein the osmotic pump tablet further comprises a first layer comprising the first composition, a second layer comprising the second composition, and a semipermeable coating, wherein the first layer further comprises a polymeric osmogen, and wherein the osmotic pump tablet is configured such that the second agent is released through burst release and the first agent is released through leaching through the semipermeable coating when the osmotic pump tablet is contacted with an aqueous solution.

19. A method for treating a subject having a brain or behavioral health disorder, an anxiety disorder, or a method of increasing advancement of a subject along Maslow's hierarchy of needs, comprising administering to the subject the formulation of claim 1, thereby treating the brain or behavioral health disorder.

20. A kit comprising the formulation of claim 1, for the treatment of a brain and/or behavioral health disorder or a symptom thereof.
